(19) United States
(12) Patent Application Publication
(10) Pub. No.: US 2007/0218512 A1
(43) Pub. Date: Sep. 20, 2007

(54) METHODS RELATED TO MMP26 STATUS AS A DIAGNOSTIC AND PROGNOSTIC TOOL IN CANCER MANAGEMENT

(76) Inventors: Alex Strongin, LaJolla, CA (US); Stan Krajewski, LaJolla, CA (US); Maryla Krajewski, LaJolla, CA (US); Alexei Savinov, LaJolla, CA (US)

Correspondence Address: NEEDLE & ROSENBERG, P.C., SUITE 1000, 999 PEACHTREE STREET, ATLANTA, GA, 30309-3915, US

(21) Appl. No.: 11/680,396

(22) Filed: Feb. 28, 2007

Related U.S. Application Data
(60) Provisional application No. 60/778,081, filed on Feb. 28, 2006.

(51) Int. Cl. G01N 33/574

(52) U.S. Cl. 435/7.23

(57) ABSTRACT

Disclosed herein are compositions and methods involving identifying MMP-26 in a subject with cancer.

### CROSS-REFERENCE TO RELATED APPLICATIONS

This application claims benefit of U.S. Provisional Application No. 60/778,081, filed Feb. 28, 2006, which is hereby incorporated herein by reference in its entirety.

### STATEMENT REGARDING FEDERALLY SPONSORED RESEARCH

This invention was made with government support under Grants 5RO1-NS36821, RO1-CA77470, U54—RR020843 and RO1-CA83017 awarded by National Institutes of Health. The government has certain rights in the invention.

### BACKGROUND

Breast cancer is the most common malignancy in Western women, and it is second only to lung cancer as the most common cause of cancer death. It affects millions of women worldwide. The therapeutic options for the treatment of breast cancers are complex and varied, including surgery, radiotherapy, endocrine therapy, and cytotoxic chemotherapy.

Roughly 75% of breast cancers are positive for the hormone-based estrogen receptor (ER) and/or progesterone receptor (PGR). Most of these patients are treated with an endocrine therapy, either as an adjuvant to surgery in early stage disease or as the primary treatment in more advanced disease. The most common endocrine therapy has been the selective estrogen receptor modulator (SERM) tamoxifen (Nolvadex). It has been in use for over 20 years and demonstrably prolongs survival.

Recent studies in post-menopausal women have demonstrated the effectiveness of a different class of endocrine therapy drugs, aromatase inhibitors. In contrast to tamoxifen, which competes with estrogen for binding to ER, aromatase inhibitors directly reduce circulating estrogen levels. Thus, patients who might be resistant to tamoxifen due to its agonist characteristics arising from cross-talk with other growth pathways or deregulation of ER coregulators might be sensitive to aromatase inhibitors. Aromatase inhibitors provide longer recurrence-free survival and generally lower risk of endometrial cancer and thromboembolic events. However, improvements in overall survival are not yet clear, and treatments are accompanied by a different set of side effects, including bone fracture risk and arthralgia. Additionally, the long-term consequences of their use are currently unknown, and the treatments are currently quite costly and only recommended in postmenopausal women. Thus, tamoxifen will remain important in adjuvant breast cancer therapy. Accurate treatment outcome prediction could guide patients to the most biologically and cost effective treatments in a timely fashion.

Intense research has been conducted in recent years on molecular markers that can provide prognostic information and/or predict treatment outcome. The standard hormone receptors (ER and PGR), as well as the growth factor receptors EGFR and ERBB2, are often used in this regard. In addition, the tumor suppressors CDKN1B and TP-53, the anti-apoptotic factor BCL2, the proliferation markers CCND1 and KI-67, and the MYC oncogene have been used for this purpose. However, the art lacks a reliable and robust test for diagnosing and prognosing of breast cancer.

Thus, needed in the art are reliable methods for both diagnosing, prognosing, and treating cancer, as well as predicting treatment outcomes.

### BRIEF SUMMARY

In accordance with the purpose of this invention, as embodied and broadly described herein, this invention relates to diagnosis, prognosis, and treatment of cancer.

Disclosed herein is a method for evaluating the prognosis of a subject with cancer, the method comprising detecting a biomarker comprising MMP-26 in the subject, wherein the presence, level, amount, or a combination, of MMP-26 is indicative of the prognosis of the subject.

Also disclosed is a method for predicting a response of a subject with cancer to a selected treatment, the method comprising detecting a biomarker comprising MMP-26 in the subject, wherein the presence, level, amount, or a combination, of MMP-26 is indicative of a given response to the selected treatment, thereby predicting the response of the subject with cancer to the selected treatment.

Further disclosed is a method of predicting the likelihood of survival of a subject with cancer comprising detecting a biomarker comprising MMP-26 in the subject, wherein the presence, level, amount, or a combination, of MMP-26 is indicative of the likelihood of survival.

Disclosed herein is a method of treating cancer in a subject, the method comprising: identifying the presence of a biomarker comprising MMP-26 in a subject; determining treatment type based on the presence of MMP-26 in a subject; and treating a subject according to the results of the previous step.

Further disclosed is a method of determining the effectiveness of an anti-cancer therapy comprising: obtaining a sample from a subject undergoing anti-cancer therapy, and monitoring the sample for expression of MMP-26, whereby expression of MMP-26 indicates the effectiveness of the anti-cancer therapy.

Also disclosed are kits comprising an assay for measuring MMP-26 levels in a subject.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows MMP-26 cleaves the N-terminal A/B domain of ERβ. (A) Upper panels—AAT is comparably sensitive to proteolysis by MT1-MMP and MMP-26. AAT was incubated with increasing amounts of MMP-26 and MT1-MMP to generate the 55 kDa cleavage fragment. Bottom panels—MMP-26 cleaves ERβ (59 kDa), but not ERα (64 kDa), while ERβ is resistant to the in vitro cleavage by MT1-MMP. The digest samples were separated by SDS-PAGE. The gels were stained with Coomassie to visualize the cleavage fragments. (B) The MMP-26 cleavage fragment represents the C-terminal part of ERβ. Following co-incubation with MMP-26, the digest samples were analyzed by Western blotting with the antibodies 14C8 and AB1410 to the N-terminal part of ERβ (right and central panels, respectively) and the antibody Ab-24 to the C-terminal part of ERβ (left panel). (C) MMP-26 and the cleavage of ERβ. MMP-26 is an estrogen-inducible gene (6) and the expression of cellular MMP-26 requires the presence of ERα/ERβ in the cells. Estrogen, through either ERα or ERβ or both, induces the expression of cellular MMP-26. MMP-26 cleaves the N-terminal sequence of ERβ (59 kDa) and this cleavage generates the C-terminal fragments (51-54 kDa) of the receptor. The 1-148 N-terminal sequence represents the A/B transactivation domain of ERβ. The shaded boxes represent the relative positions of the epitopes of the AB1410 (raised against the 1-12 N-terminal sequence of ERβ), 14C8 (raised against the 1-150 N-terminal sequence of ERβ) and Ab-24 (raised against the C-terminal part ERβ) in the ERβ sequence.

FIG. 2 shows the presence of MMP-26 correlates with the proteolysis of ERβ in endometrial carcinoma Ishikawa cells and breast carcinoma MCF-7 cells. (A) Left panel—Western blotting of MMP-26 naturally expressed by Ishikawa cells and the purified MMP-26 control. Right panel—Western blotting of ERβ expressed by Ishikawa and MCF-7 cells, intact ERα and ERβ co-incubated with MMP-26. (B) Left panel—Western blotting of MMP-26 from MCF-7 cells transfected with the control lentiviral vector (mock) and the lentivirus bearing MMP-26 (MMP-26), and the purified MMP-26 control. Right panel—Western blotting of ERβ from mock- and MMP-26-transfected MCF-7 cells, intact ERβ and ERβ co-incubated with MMP-26.

FIG. 3 shows immunostaining of Ishikawa and MCF-7 cells. Immunoreactivity was evident in Ishikawa cells stained with the MMP-26 antibody and the ERβ antibody AB 1410. There was no ERα immunoreactivity in Ishikawa cells. Immunofluorescence staining confirms the expression of MMP-26 and ERβ (AB1410 antibody) in MCF-7 cells. The staining with the antibody control was negative.

FIG. 4 shows immunohistochemical analysis of MMP-26 and ERβ in archival breast cancer biopsies. (A) Distribution of MMP-26 immunostaining in normal epithelium (NE), ductal carcinoma in situ (DCIS) and invasive carcinoma (invasive). The box and the whiskers (bars) indicate ±SEM and ±standard deviation (SED), respectively. A mean value is plotted as the middle bar. P=0.000003; ANOVA. (B) Correlation of the MMP-26 expression with the clinical stage of breast cancer. The box and the whiskers (bars) indicate ±SEM and ±standard deviation (SED), respectively. A mean value is plotted as the middle point for the early, I-II, stage and the late, III, stage breast carcinomas. P=0.01; ANOVA. (C) Cumulative survival curves (Kaplan-Meier survival analysis) for MMP-26 expression-positive DCIS. Low MMP-26 expression means the immunoscore <60; high MMP-26 expression indicates the immunoscore >60. The immunostaining data were dichotomized according to median immunoscore. P=0.04 is the significance of the log rank. (D) Inverse correlation of MMP-26 with ERβ detected by immunohistochemistry. Scatter diagram of MMP-26 and ERβ immunoscores shows the linear regression line. The negative correlation is measured by the correlation coefficient (r=−0.22). P=0.01; ANOVA. (E) Cumulative survival curves (Kaplan-Meier survival analysis) for ERβ expression in the ERα-positive breast cancer patient cohort. Low ERβ expression means the immunoscore <80; high ERβ expression indicates the immunoscore >80. p=0.04 is the significance of the log rank.

FIG. 5 shows representative immunostaining of ERα, ERβ and MMP-26 in invasive ductal carcinomas. TMAs were stained (DAB, brown) with the antibody 1D5 to ERα (panels B and E) and with the antibody Ab-24 to the C-terminal portion of ERβ (panels C and F). In double-labeling staining, TMAs were stained with the antibody AB1410 to the N-terminal portions of ERβ (DAB, brown; panels A and D) and with the antibody to MMP-26 (SR chromagen, grey/black). TMAs were counterstained with Nuclear Red (pink). The bottom portions of panels A-F (×60 magnification) show the respective enlarged images (×250 magnification).

FIG. 6 shows representative immunostaining of ERα, ERβ and MMP-26 in DCIS. TMAs were stained (DAB, brown) with the antibody 1D5 to ERα (panels B and E) and with the antibody Ab-24 to the C-terminal portion of ERβ (panels C and F). In double-labeling staining, TMAs were stained with the antibody AB1410 to the N-terminal portions of ERβ (DAB, brown; panels A and D) and with the antibody to MMP-26 (SR chromagen, grey/black). TMAs were counterstained with Nuclear Red (pink). The bottom portions of panels A-F (×60 magnification) show the respective enlarged images (×250 magnification).

### DETAILED DESCRIPTION

The disclosed methods and compositions related thereto may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a compound is disclosed and discussed as a treatment method, each and every combination of this compound and other compounds and compositions that can be used for treatment are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed methods are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Human matrix metalloproteinases (MMPs) are a family of twenty-four zinc-enzymes that degrade the extracellular matrix and cell surface molecules (Egeblad et al. Nat Rev Cancer 2002 2:161-74). The prodomain of all MMPs exhibits the sequence motif PRCG called the “cysteine-switch” (Nagase et al. J Biol Chem 1999 274:21491-4). An unpaired Cys sulfhydryl group of the PRCG cysteine-switch binds the active site zinc. The Cys-Zn interactions are essential for maintaining the latency of MMP zymogens. There is, however, an exception from this general rule. An unconventional PH₈₁CGVPD cysteine switch distinguishes human MMP-26 from other members of the MMP superfamily (Zhao et al. J Biol Chem 2003 278:15056-64; Park et al. J Biol Chem 2002 277:35168-75; Marchenko et al. J Biol Chem 2002 277:18967-72). The presence of the His-81 in the immediate proximity of the Cys-82 residue, in addition to other atypical structural features, leads to the unorthodox, autolytic mechanisms of the MMP-26 zymogen activation and contributes to the unusual physiological role of MMP-26 in cells and tissues (Zhao et al. J Biol Chem 2003 278:15056-64; Li et al. Cancer Res 2004 64:8657-65; Yamamoto et al. Carcinogenesis 2004 25:2353-60; Marchenko et al. Int J Biochem Cell Biol 2004 36:942-56; Goffin et al. Biol Reprod 2003 69:976-84; Marchenko et al. Biochem J 2001 356:705-18; Uria et al. Cancer Res 2000 60:4745-51; de Coignac et al. Eur J Biochem 2000 267 3323-9; Park et al. Biol Chem 2000 275:20540-4). In contrast with other MMPs, which are either secretory, soluble, or membrane-anchored enzymes, MMP-26 primarily accumulates in the intracellular milieu (Park et al. J Biol Chem 2002 277:35168-75; Marchenko et al. Int J Biochem Cell Biol 2004 36:942-56; Isaka et al. Cancer 2003 97:79-89).

The promoter of the MMP-26 gene includes the estrogen-response element (ERE) that binds estrogen receptors (ERs) (Li et al. Cancer Res 2004 64:8657-65). Estrogens, primarily 17β-estradiol (E2), signaling is transmitted by ERs. ERs are members of a nuclear receptor superfamily, and are encoded by two distinct genes, ERα and ERβ (Gustafsson J Endocrinol 1999 163:379-83). Five ERβ isoforms, which diverge at a common position within the predicted helix 10 of the ligand-binding domain, have been identified and cloned (Tong et al. Breast Cancer Res Treat 2002 71:249-55). This work was performed with the ERβ1 isoform, which was termed ERβ for the clarity of presentation.

ERs consist of five individual domains: the N-terminal A/B domain with a 16% sequence identity between the two ERs, the highly conserved central DNA-binding domain (DBD; 96% sequence identity), the flexible hinge D domain (D; 30% sequence identity), the ligand-binding domain (LBD; 59% sequence identity) and the C-terminal, short, F domain (18% sequence identity). The A/B domain is responsible for the ligand-independent transactivation function (AF-1). The D domain contains a nuclear localization signal. The multifunctional LBD domain, in addition to its role in ligand binding, is involved in dimerization and the ligand-dependent transactivation function (AF-2) (Pettersson et al. Annu Rev Physiol 2001 63:165-92).

The E2-ER complex stimulates, via the binding of the ERE motif, the transcriptional activity of the MMP-26 gene promoter in hormone-regulated neoplasms, including breast, ovarian and endometrial carcinomas as well as the normal reproductive processes and menstr (Li et al. Cancer Res 2004 64:8657-65; Chegini et al. Fertil Steril 2003 80:564-70; Pilka et al. Mol Hum Reprod 2003 9:271-7; Li et al. Mol Hum Reprod 2002 8:934-40; Marchenko et al. Biochem J 2002 363:253-62)ual cycle. Using immunohistochemical analysis, it was determined, however, that an inverse correlation, rather than a direct correlation, frequently occurs between the levels of MMP-26 and ER in biopsy samples from breast cancer patients. These findings prompted the finding that there is a regulatory loop in hormone-regulated malignancies and that this loop links E2-induced MMP-26 to the proteolysis of the ERs.

It is herein shown that the N-terminal portion of the A/B domain of ERβ was sensitive to MMP-26 proteolysis in vitro and in cell-based assays (Example 1). In the breast cancer patient cohort, the expression of MMP-26 correlated inversely with the residual levels of the intact ERβ in the adenocarcinoma specimens. Elevated MMP-26 expression DCIS was strongly associated with a longer overall survival in this patient cohort. The results show that high levels of MMP-26 in the mammary epithelium at the early stages of its malignant transformation are a marker of a favorable prognosis. Conversely, the use of broad-range MMP inhibitors such as Marimastat, which is potent against MMP-26, is not favorable for breast cancer patients, a phenomenon observed in clinical trials (Pavlaki et al. Cancer Metastasis Rev 2003 22:177-203).

### A. METHODS

1. Methods of Prognosis and Diagnosis Based on MMP-26 Status

Provided herein is a method for evaluating the prognosis of a subject with cancer, the method comprising detecting a biomarker comprising MMP-26 in the subject, wherein the presence, level, amount, or a combination, of MMP-26 is indicative of the prognosis of the subject.

The term “prognosis” encompasses predictions about the likely course of disease or disease progression, particularly with respect to likelihood of disease remission, disease relapse, tumor recurrence, metastasis, and death. “Good prognosis” refers to the likelihood that a patient afflicted with cancer, particularly breast cancer, will remain disease-free (i.e., cancer-free). “Poor prognosis” is intended to mean the likelihood of a relapse or recurrence of the underlying cancer or tumor, metastasis, or death. Cancer patients classified as having a “good outcome” remain free of the underlying cancer or tumor. In contrast, “bad outcome” cancer patients experience disease relapse, tumor recurrence, metastasis, or death. In particular embodiments, the time frame for assessing prognosis and outcome is, for example, less than one year, one, two, three, four, five, six, seven, eight, nine, ten, fifteen, twenty or more years. As used herein, the relevant time for assessing prognosis or disease-free survival time begins with the surgical removal of the tumor or suppression, mitigation, or inhibition of tumor growth. Thus, for example, in particular embodiments, a “good prognosis” refers to the likelihood that a breast cancer patient will remain free of the underlying cancer or tumor for a period of at least five, more particularly, a period of at least ten years. In further aspects of the invention, a “bad prognosis” refers to the likelihood that a breast cancer patient will experience disease relapse, tumor recurrence, metastasis, or death within less than five years, more particularly less than ten years. Time frames for assessing prognosis and outcome provided above are illustrative and are not intended to be limiting.

In one example, prognostic performance of the MMP-26 biomarker, and/or other biomarkers and clinical parameters can be assessed utilizing a Cox Proportional Hazards Model Analysis, which is a regression method for survival data that provides an estimate of the hazard ratio and its confidence interval. The Cox model is a well-recognized statistical technique for exploring the relationship between the survival of a subject and particular variables. This statistical method permits estimation of the hazard (i.e., risk) of individuals given their prognostic variables (e.g., overexpression of particular biomarkers, as described herein). Cox model data are commonly presented as Kaplan-Meier curves. The “hazard ratio” is the risk of death at any given time point for subjects displaying particular prognostic variables. See generally Spruance et al. (2004) Antimicrob. Agents & Chemo. 48:2787-2792. For example, the MMP-26 biomarker is statistically significant for assessment of the likelihood of breast cancer recurrence or death due to the underlying breast cancer. Methods for assessing statistical significance are well known in the art and include, for example, using a log-rank test Cox analysis and Kaplan-Meier curves. In one example, a p-value of less than 0.05 constitutes statistical significance.

The cancer evaluated can be breast cancer. The breast cancer can be ERα/β-positive. By “breast cancer” is intended, for example, those conditions classified by biopsy as malignant pathology. The clinical delineation of breast cancer diagnoses is well-known in the medical arts. One of skill in the art will appreciate that breast cancer refers to any malignancy of the breast tissue, including, for example, carcinomas and sarcomas. In particular embodiments, the breast cancer is ductal carcinoma in situ (DCIS), lobular carcinoma in situ (LCIS), or mucinous carcinoma. Breast cancer also refers to infiltrating ductal (IDC) or infiltrating lobular carcinoma (ILC).

The level of MMP-26 can be measured in the subject. This can be done in a variety of ways, as disclosed below. Higher levels of MMP-26 can indicate a good prognosis. The stage of cancer can also be taken into consideration when determining prognosis, along with other factors such as other biomarkers or clinical information, described below. As disclosed above, the level of MMP-26 can be compared to a reference level, wherein the magnitude and direction of a difference between the level of MMP-26 and the reference level is indicative of the prognosis of the subject. The prognosis of the subject can be used to determine disease progression in the subject as well. Therefore, the breast cancer stage can be used both in conjunction with the MMP-26 status, and can also be adjusted according to the MMP-26 status.

The American Joint Committee on Cancer (AJCC) has developed a standardized system for breast cancer staging using a “TNM” classification scheme. Patients are assessed for primary tumor size (T), regional lymph node status (N), and the presence/absence of distant metastasis (M) and then classified into stages 0-IV based on this combination of factors. In this system, primary tumor size is categorized on a scale of 0-4 (T0=no evidence of primary tumor; T1=<2 cm; T2=>2 cm <5 cm; T3=>5 cm; T4=tumor of any size with direct spread to chest wall or skin). Lymph node status is classified as N0-N3 (N0=regional lymph nodes are free of metastasis; N1=metastasis to movable, same-side axillary lymph node(s); N2=metastasis to same-side lymph node(s) fixed to one another or to other structures; N3=metastasis to same-side lymph nodes beneath the breastbone). Metastasis is categorized by the absence (M0) or presence of distant metastases (M1). While cancer subjects at any clinical stage are encompassed by the methods disclosed herein, breast cancer patients in early-stage breast cancer are of particular interest. By “early-stage breast cancer” is intended stages 0 (in situ breast cancer), I (T1, N0, M0), IIA (T0-1, N1, M0 or T2, N0, M0), and IIB (T2, N1, M0 or T3, N0, M0). Early-stage breast cancer patients exhibit little or no lymph node involvement. As used herein, “lymph node involvement” or “lymph node status” refers to whether the cancer has metastasized to the lymph nodes. Breast cancer patients are classified as “lymph node-positive” or “lymph node-negative” on this basis. Methods of identifying breast cancer patients and staging the disease are well known and may include manual examination, biopsy, review of patient's and/or family history, and imaging techniques, such as mammography, magnetic resonance imaging (MRI), and positron emission tomography (PET).

As mentioned above, the presence of MMP-26 in a subject is most favorable in early-stage breast cancer. When a subject has early stage breast cancer, or DCIS, the prognosis is generally considered good when MMP-26 is present. However, other factors can also be taken into consideration when making this assessment, such as clinical information and the presence or absence, and expression levels, of other biomarkers.

Various clinical information about the subject can be analyzed to help determine the prognosis of the subject. For example, the clinical information comprises tumor size, tumor grade, lymph node status, age, menopause status, chance of recurrence, disease free and overall survival rate, applied therapy strategy, status of ERα, PR and Her-2/neu status, and family history.

The method for evaluating the prognosis of a subject with breast cancer can further comprise assessing one or more additional biomarkers in the subject. These biomarkers can also be assessed in conjunction with the other methods disclosed herein. Examples of such biomarkers include, but are not limited to, MYC, RB1, TP53, ATM, BAX, BRCA1, BRCA2, EGFR, ESR1, NME1, PTEN, BCL2, CCND1, CCNE1, CDK4, FGF3, FGF8, IGF2, MAPK3, PRKCA, TGFA, TGFB1, TGFB2, TGFB3, VEGF, CDK2, EGF, PCNA, BMP6, CSF1, CSF3, FGF18, TNF, IGF1, ODZ1, PLG, ESR2, IGFBP3, TSG101, AR, ERBB2, ERBB4, PRKD1, PRL, MX1, PRKCE, AKTI, BAG3, BCL2L1, PRKCZ, RAD51, XRCC3, CD34, CDH1, CTNNB1, ITGB3, PECAM1, ALB, COL4A2, INS, KLK13, MMP11, MMP9, SERPINE1, SHBG, ERBB3, PDPK1, PRKCB1, PRKCD, PRKCG, PRKCZ, PRKD2, SRC, TYK2, EGR3, FOS, JUN, NR4A1, PGR, SP1, CTSB, CTSC, CTSD, CTSE, CTSL2, PCSK6, ABCB1, ABCG2, AKAP1, CEACAM5, CYB5, CYC1, CYP19A1, GSTM1, GSTM3, KRT19, MIB1, MUC1, MUC19, VIM, CCNE2, EXT1, CCNB1, CCNB2, CDC25B, CENPF, MKI67, MYBL2, PCTK1, PSMD2, MCM6, ORC6L, RFC4, RRM2, BIRC5, CKS2, MAD2L1, SMC4L1, STK6, ESM1, FLT1, BTG2, CHPT1, IGFBP5, WISP1, BUB1, CKS2, MAPRE2, MKI67, NDRG1, BAG1, BIRC5, BNIP3, RAD21, STK3, ADM, CP, MATN3, RBP3, TFRC, CDC42BPA, CKS2, MELK, STK3, STK32B, MTMR2, EZH2, HMGB3, IVNS1ABP, KIAA1442, MCM6, MLLT10, PIR, SEC14L2, TBX3, TRIP13, BIRC5, GGH, PITRM1, UCHL5, ACADS, ALDH4A1, ALDH6A1, AP2B1, ASNS, ASPM, BBC3, BM039, C20orf103, C20orf28, C20orf46, CA9, CD68, CENPA, CIRBP, CTPS, DCK, DEGS, DEPDC1, DKFZP434B168, DKFZp762E1312, DLG7, ECT2, EGLN1, EIF2C2, ERP70, EVL, FBP1, FBXO31, FBXO5, FGD6, FLJ10134, FLJ10156, FLJ10511, FLJ10901, FLJ12150, FLJ21924, FLJ22341, FUT8, GBE1, GCNlL1, GMPS, GNAZ, GPR126, GPSM2, GRB7, HRASLS, HRB, 1HPK2, ITR, KIAA0882, KIAA1181, KIAA1217, KIAA1324, KIAA1683, KIF14, KIF21A, KIF3B, KNTC2, KRT18, LCHN, LGP2, LOC388134, LOC56901, LYRIC, M160, MCCC1, MGAT4A, MIR, MLF1IP, MRPL13, MS4A7, MYRIP, NMB, NMU, NUSAP1, ODZ3, OXCT, PALM2-AKAP2, PAQR3, PECI, PEX12, PFKP, PGK1, PIB5PA, PLEKHA1, PRAME, PRC1, PRO2000, PSMD7, PTDSS1, PTPLB, QDPR, RAB27B, RAB6B, RAI2, RAMP, RASL11B, RPS4X, RRAGD, SACS, SCUBE2, SERF1A, SLC2A3, SLC7A1, Spc25, ST7, STMN1, STX1A, SYNCRIP, TK1, TMEFF1, ER-β cleavage products, and caspase-14.

Also disclosed are methods for predicting a response of a subject with cancer to a selected treatment, the method comprising detecting a biomarker comprising MMP-26 in the subject, wherein the presence, level, amount, or a combination, of MMP-26 is indicative of a given response to the selected treatment, thereby predicting the response of the subject with cancer to the selected treatment.

Also disclosed is a method of treating cancer in a subject, the method comprising: identifying the presence of a biomarker comprising MMP-26 in a subject; determining treatment type based on the presence of MMP-26 in a subject; and treating a subject according to the results of the determining step.

As mentioned above, MMP-26 status can help determine what strategy to take in treatment. In one example, the use of broad-range MMP inhibitors such as Marimastat can be avoided in subjects in which MMP-26 is detected. Examples of treatment methods include surgery, radiation therapy, hormone therapy, chemotherapy, or some combination thereof. As is known in the art, treatment decisions for individual breast cancer subjects can be based on the number of lymph nodes involved, estrogen and progesterone receptor status, size of the primary tumor, and stage of the disease at diagnosis. Current treatment strategies can be found, for example, the University of Texas MD Anderson Cancer Center Breast Invasive Cancer Treatment Guidelines (2005), which is herein incorporated by reference in its entirety. This guide provides detailed information, including a decision tree, based on various factors. Provided are guidelines on both invasive and non-invasive forms of breast cancer. Analysis of a variety of clinical factors and clinical trials has also led to the development of recommendations and treatment guidelines for early-stage breast cancer by the International Consensus Panel of the St. Gallen Conference (2001). See Goldhirsch et al. (2001) J. Clin. Oncol. 19:3817-3827, which is herein incorporated by reference in its entirety. The guidelines indicate that treatment for patients with node-negative breast cancer varies substantially according to the baseline prognosis. More aggressive treatment is recommended for patients with a relative high risk of recurrence when compared to patients with a relatively low risk of recurrence. It has been demonstrated that chemotherapy for the high risk population has resulted in a reduction in the risk of relapse. Women with a low risk category are usually treated with radiation and hormonal therapy. Stratification of patients into poor prognosis or good prognosis risk groups at the time of diagnosis using the methods disclosed herein may provide an additional or alternative treatment decision-making factor. The methods disclosed herein permit the differentiation of breast cancer patients with a good prognosis from those more likely to suffer a recurrence (i.e., patients who might need or benefit from additional aggressive treatment at the time of diagnosis).

The methods disclosed herein find particular use in choosing appropriate treatment for early-stage breast cancer patients. The majority of breast cancer patients diagnosed at an early-stage of the disease enjoy long-term survival following surgery and/or radiation therapy without further adjuvant therapy. A significant percentage (approximately 20%) of these patients, however, will suffer disease recurrence or death, leading to clinical recommendations that some or all early-stage breast cancer patients should receive adjuvant therapy (e.g., chemotherapy). The methods disclosed herein find use in identifying this high-risk, poor prognosis population of early-stage breast cancer patients and thereby determining which patients would benefit from continued and/or more aggressive therapy and close monitoring following treatment. For example, early-stage breast cancer patients assessed as having a poor prognosis by the methods disclosed herein (such as the lack of MMP-26, for example) may be selected for more aggressive adjuvant therapy, such as chemotherapy, following surgery and/or radiation treatment. In particular embodiments, the methods of the present invention may be used in conjunction with the treatment guidelines established by the St. Gallens Conference to permit physicians to make more informed breast cancer treatment decisions. The present methods for evaluating breast cancer prognosis can also be combined with other prognostic methods and molecular marker analyses known in the art for purposes of selecting an appropriate breast cancer treatment. Furthermore, the methods disclosed herein can be combined with later-developed prognostic methods and molecular marker analyses not currently known in the art.

For example, patients who have been diagnosed as having stage 0 or stage 1 breast cancer that are considered MMP-26 negative can be treated more aggressively, as disclosed above. Alternatively, those patients with a stage 0 or stage 1 breast cancer designation that are positive for MMP-26 can be counseled to take a “wait and watch” approach. For example, a positive MMP-26 test can support a “wait and watch approach” for subjects with DCIS and Stage 0, T is N0 M0. This can also help determine how aggressively treat by surgery and with Tamoxifen™.

Also disclosed is a method of determining the effectiveness of an anti-cancer therapy comprising obtaining a sample from a subject undergoing anti-cancer therapy, and monitoring the sample for expression of MMP-26, whereby expression of MMP-26 indicates the effectiveness of the anti-cancer therapy. In one example, the level of expression of MMP-26 is compared with a previous sample taken from the same subject. The level of expression of MMP-26 can also be compared with a standard level, wherein increasing levels of MMP-26 indicates an effective anti-cancer therapy. Examples of anti-cancer therapies are given above.

Also disclosed herein is a method of predicting the likelihood of survival of a subject with cancer comprising detecting a biomarker comprising MMP-26 in the subject, wherein the presence, level, amount, or a combination, of MMP-26 is indicative of the likelihood of survival. In particular, the methods may be used to predict the likelihood of long-term, disease-free survival. By “predicting the likelihood of survival of a subject with cancer” is intended assessing the risk that a subject will die as a result of the underlying breast cancer. “Long-term, disease-free survival” is intended to mean that the subject does not die from or suffer a recurrence of the underlying breast cancer within a period of at least five years, more particularly at least ten or more years, following initial diagnosis or treatment. Such methods for predicting the likelihood of survival of a breast cancer patient comprise detecting expression of MMP-26 in a subject sample, wherein the likelihood of survival, particularly long-term, disease-free survival, decreases as the number of biomarkers determined to be overexpressed in the patient sample increases. Other aspects can also be taken into account when assessing the likelihood of survival, such as other the expression of other biomarkers and clinical information, as discussed herein. Likelihood of survival can be assessed in comparison to, for example, breast cancer survival statistics available in the art.

2. Detecting MMP-26

Methods for detecting expression of MMP-26 can comprise any methods that determine the quantity or the presence of the biomarkers either at the nucleic acid or protein level. Such methods are well known in the art and include but are not limited to western blots, northern blots, southern blots, ELISA, immunoprecipitation, immunofluorescence, flow cytometry, immunohistochemistry, nucleic acid hybridization techniques, nucleic acid reverse transcription methods, and nucleic acid amplification methods. In particular embodiments, expression of a biomarker is detected on a protein level using, for example, antibodies that are directed against specific biomarker proteins. These antibodies can be used in various methods such as Western blot, ELISA, immunoprecipitation, or immunohistochemistry techniques. Likewise, immunostaining of breast tissue, particularly breast tumor tissue, can be combined with assessment of clinical information, conventional prognostic methods, and expression of molecular markers known in the art, such as those disclosed below. In this manner, the disclosed methods can permit the more accurate determination of breast cancer prognosis.

Any methods available in the art for detecting expression of biomarkers are encompassed herein. The expression of MMP-26 can be detected on a nucleic acid level or a protein level. By “detecting expression” is intended determining the quantity or presence of a biomarker gene or protein. Thus, “detecting expression” encompasses instances where a biomarker is determined not to be expressed, not to be detectably expressed, expressed at a low level, expressed at a normal level, or overexpressed. In order to determine overexpression, the sample to be examined may be compared with a corresponding sample that originates from a healthy person. That is, the “normal” level of expression is the level of expression of the biomarker in, for example, a breast tissue sample from a human subject or patient not afflicted with breast cancer. Such a sample can be present in standardized form. In some embodiments, determination of biomarker overexpression requires no comparison between the sample and a corresponding sample that originates from a healthy person. For example, detection of expression of the MMP-26 biomarker, which is indicative of a good prognosis in a breast tumor sample may preclude the need for comparison to a corresponding breast tissue sample that originates from a healthy person. Moreover, no expression, underexpression, or normal expression (i.e., the absence of overexpression) of a biomarker or combination of biomarkers of interest provides useful information regarding the prognosis of a breast cancer subject.

By “sample” is intended any sampling of cells, tissues, or bodily fluids in which expression of the MMP-26 biomarker can be detected. Examples of such samples include but are not limited to blood, lymph, urine, gynecological fluids, biopsies, and smears. Bodily fluids useful in the present invention include blood, urine, saliva, nipple aspirates, or any other bodily secretion or derivative thereof. Blood can include whole blood, plasma, serum, or any derivative of blood. In preferred embodiments, the sample comprises breast cells, particularly breast tissue from a biopsy, more particularly a breast tumor tissue sample. However, the sample need not comprise breast tissue, and can be obtained from normal tissue, fluid, or cells. Samples may be obtained from a subject by a variety of techniques including, for example, by scraping or swabbing an area, by using a needle to aspirate bodily fluids, or by removing a tissue sample (i.e., biopsy). Methods for collecting various samples are well known in the art. In some embodiments, a breast tissue sample is obtained by, for example, fine needle aspiration biopsy, core needle biopsy, or excisional biopsy. Fixative and staining solutions may be applied to the cells or tissues for preserving the specimen and for facilitating examination. Body samples, particularly breast tissue samples, may be transferred to a glass slide for viewing under magnification. In preferred embodiments, the body sample is a formalin-fixed, paraffin-embedded breast tissue sample, particularly a primary breast tumor sample.

i. Antibody Detection/Immunohistochemistry

An immunohistochemistry technique can be used for evaluating the prognosis of a subject. Specifically, this method comprises antibody staining of the MMP-26 biomarker. One of skill in the art will recognize that the immunohistochemistry methods described herein below may be performed manually or in an automated fashion using, for example, the Autostainer Universal Staining System (Dako™).

In one immunohistochemistry method, a tissue sample is collected by, for example, biopsy techniques known in the art. Samples may be frozen for later preparation or immediately placed in a fixative solution. Tissue samples may be fixed by treatment with a reagent such as formalin, gluteraldehyde, methanol, or the like and embedded in paraffin. Methods for preparing slides for immunohistochemical analysis from formalin-fixed, paraffin-embedded tissue samples are well known in the art.

In one example, determining MMP-26 status can comprise collecting a sample, contacting the sample with at least one antibody specific for a biomarker of interest, detecting antibody binding, and determining if the biomarker is expressed. That is, samples are incubated with the biomarker antibody for a time sufficient to permit the formation of antibody-antigen complexes, and antibody binding is detected, for example, by a labeled secondary antibody. Samples are classified as having a good or a poor prognosis based on the level of MMP-26 detected, or merely the presence or absence of MMP-26, as defined below. The definition of “good” and “poor” prognosis, and the factors which go into determining such, as discussed in more detail elsewhere herein as well.

As used herein, “antigen retrieval” or “antigen unmasking” refers to methods for increasing antigen accessibility or recovering antigenicity in, for example, formalin-fixed, paraffin-embedded tissue samples. Any method for making antigens more accessible for antibody binding may be used in the practice of the invention, including those antigen retrieval methods known in the art. See, for example, Hanausek and Walaszek, eds. (1998) Tumor Marker Protocols (Humana Press, Inc., Totowa, N.J.); and Shi et al., eds. (2000) Antigen Retrieval Techniques: Immunohistochemistry and Molecular Morphology (Eaton Publishing, Natick, Mass.), both of which are herein incorporated by reference in their entirety.

Antigen retrieval methods include but are not limited to treatment with proteolytic enzymes (e.g., trypsin, chymoptrypsin, pepsin, pronase, etc.) or antigen retrieval solutions. Antigen retrieval solutions of interest include, for example, citrate buffer, pH 6.0 (Dako™), tris buffer, pH 9.5 (Biocare™), EDTA, pH 8.0 (Biocare™), L.A.B. (“Liberate Antibody Binding Solution;” Polysciences), antigen retrieval Glyca solution (Biogenex™), citrate buffer solution, pH 4.0 (Zymed™), Dawn™ detergent (Proctor & Gamble™), deionized water, and 2% glacial acetic acid. In some embodiments, antigen retrieval comprises applying the antigen retrieval solution to a formalin-fixed tissue sample and then heating the sample in an oven (e.g., 60° C.), steamer (e.g., 95° C.), or pressure cooker (e.g., 120° C.) at specified temperatures for defined time periods. In other aspects, antigen retrieval may be performed at room temperature. Incubation times will vary with the particular antigen retrieval solution selected and with the incubation temperature. For example, an antigen retrieval solution may be applied to a sample for as little as 5, 10, 20, or 30 minutes or up to overnight. The design of assays to determine the appropriate antigen retrieval solution and optimal incubation times and temperatures is standard and well within the routine capabilities of those of ordinary skill in the art.

Following antigen retrieval, samples are blocked using an appropriate blocking agent, e.g., hydrogen peroxide. An antibody directed to MMP-26 is then incubated with the sample for a time sufficient to permit antigen-antibody binding. As noted above, one of skill in the art will appreciate that a more accurate breast cancer prognosis may be obtained in some cases by detecting overexpression of more than one biomarker in a subject. Therefore, in particular embodiments, at least two antibodies directed to two distinct biomarkers are used to evaluate the prognosis of a breast cancer patient. Where more than one antibody is used, these antibodies may be added to a single sample sequentially as individual antibody reagents or simultaneously as an antibody cocktail. Alternatively, each individual antibody may be added to a separate tissue section from a single patient sample, and the resulting data pooled.

Techniques for detecting antibody binding are well known in the art. Antibody binding to a biomarker of interest may be detected through the use of chemical reagents that generate a detectable signal that corresponds to the level of antibody binding and, accordingly, to the level of biomarker protein expression. For example, antibody binding can be detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include horseradish peroxidase (HRP) and alkaline phosphatase (AP). Commercial antibody detection systems, such as, for example the Dako Envision+ system™ and Biocare Medical's Mach 3™ system, may be used to practice the present invention.

In one immunohistochemistry method, antibody binding to a biomarker is detected through the use of an HRP-labeled polymer that is conjugated to a secondary antibody. Slides are stained for antibody binding using the chromogen 3,3-diaminobenzidine (DAB) and then counterstained with hematoxylin and, optionally, a bluing agent such as ammonium hydroxide. In some aspects of the invention, slides are reviewed microscopically by a pathologist to assess cell staining (i.e., biomarker overexpression) and to evaluate breast cancer prognosis. Alternatively, samples may be reviewed via automated microscopy or by personnel with the assistance of computer software that facilitates the identification of positive staining cells.

The terms “antibody” and “antibodies” broadly encompass naturally occurring forms of antibodies and recombinant antibodies such as single-chain antibodies, chimeric and humanized antibodies and multi-specific antibodies as well as fragments and derivatives of all of the foregoing, which fragments and derivatives have at least an antigenic binding site. Antibody derivatives may comprise a protein or chemical moiety conjugated to the antibody.

“Antibodies” and “immunoglobulins” (Igs) are glycoproteins having the same structural characteristics. While antibodies exhibit binding specificity to an antigen, immunoglobulins include both antibodies and other antibody-like molecules that lack antigen specificity. Polypeptides of the latter kind are, for example, produced at low levels by the lymph system and at increased levels by myelomas.

The term “antibody” is used in the broadest sense and covers fully assembled antibodies, antibody fragments that can bind antigen (e.g., Fab′, F′(ab).sub.2, Fv, single chain antibodies, diabodies), and recombinant peptides comprising the foregoing.

The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Antibody fragments” comprise a portion of an intact antibody, preferably the antigen-binding or variable region of the intact antibody. Examples of antibody fragments include Fab, Fab′, F(ab′)2, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize 35 readily. Pepsin treatment yields an F(ab′)2 fragment that has two antigen-combining sites and is still capable of cross-linking antigen.

“Fv” is the minimum antibody fragment that contains a complete antigen recognition and binding site. In a two-chain Fv species, this region consists of a dimer of one heavy- and one light-chain variable domain in tight, non-covalent association. In a single-chain Fv species, one heavy- and one light-chain variable domain can be covalently linked by flexible peptide linker such that the light and heavy chains can associate in a “dimeric” structure analogous to that in a two-chain Fv species. It is in this configuration that the three CDRs of each variable domain interact to define an antigen-binding site on the surface of the V.sub.H-V.sub.L dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H1}) of the heavy chain. Fab fragments differ from Fab′ fragments by the addition of a few residues at the carboxy terminus of the heavy-chain C_{H1} domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them.

Monoclonal antibodies can be prepared using the method of Kohler et al. (1975) Nature 256:495-496, or a modification thereof. Typically, a mouse is immunized with a solution containing an antigen. Immunization can be performed by mixing or emulsifying the antigen-containing solution in saline, preferably in an adjuvant such as Freund's complete adjuvant, and injecting the mixture or emulsion parenterally. Any method of immunization known in the art may be used to obtain the monoclonal antibodies of the invention. After immunization of the animal, the spleen (and optionally, several large lymph nodes) are removed and dissociated into single cells. The spleen cells may be screened by applying a cell suspension to a plate or well coated with the antigen of interest. The B cells expressing membrane bound immunoglobulin specific for the antigen bind to the plate and are not rinsed away. Resulting B cells, or all dissociated spleen cells, are then induced to fuse with myeloma cells to form hybridomas, and are cultured in a selective medium. The resulting cells are plated by serial dilution and are assayed for the production of antibodies that specifically bind the antigen of interest (and that do not bind to unrelated antigens). The selected monoclonal antibody (mAb)-secreting hybridomas are then cultured either in vitro (e.g., in tissue culture bottles or hollow fiber reactors), or in vivo (as ascites in mice).

As an alternative to the use of hybridomas, antibodies can be produced in a cell line such as a CHO cell line, as disclosed in U.S. Pat. Nos. 5,545,403; 5,545,405; and 5,998,144; incorporated herein by reference. Briefly the cell line is transfected with vectors capable of expressing a light chain and a heavy chain, respectively. By transfecting the two proteins on separate vectors, chimeric antibodies can be produced. Another advantage is the correct glycosylation of the antibody. A monoclonal antibody can also be identified and isolated by screening a recombinant combinatorial immunoglobulin library (e.g., an antibody phage display library) with a biomarker protein to thereby isolate immunoglobulin library members that bind the biomarker protein. Kits for generating and screening phage display libraries are commercially available (e.g., the Pharmacia Recombinant Phage Antibody System, Catalog No. 27-9400-01; and the Stratagene SurfZAP9 Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Polyclonal antibodies can be prepared by immunizing a suitable subject (e.g., rabbit, goat, mouse, or other mammal) with a biomarker protein immunogen. The antibody titer in the immunized subject can be monitored over time by standard techniques, such as with an enzyme linked immunosorbent assay (ELISA) using immobilized biomarker protein. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:55052; Kenneth (1980) in Monoclonal Antibodies: A New Dimension In Biological Analyses (Plenum Publishing Corp., NY; and Lerner (1981) Yale J. Biol. Med., 54:387-402).

Disclosed herein are monoclonal antibodies and variants and fragments thereof that specifically bind to MMP-26. The monoclonal antibodies may be labeled with a detectable substance as described below to facilitate biomarker protein detection in the sample. Such antibodies find use in practicing the methods of the invention. Monoclonal antibodies having the binding characteristics of the antibodies disclosed herein are also encompassed by the present invention. Compositions further comprise antigen-binding variants and fragments of the monoclonal antibodies, hybridoma cell lines producing these antibodies, and isolated nucleic acid molecules encoding the amino acid sequences of these monoclonal antibodies.

Antibodies having the binding characteristics of a monoclonal antibody of the invention are also provided. “Binding characteristics” or “binding specificity” when used in reference to an antibody means that the antibody recognizes the same or similar antigenic epitope as a comparison antibody. Examples of such antibodies include, for example, an antibody that competes with a monoclonal antibody of the invention in a competitive binding assay. One of skill in the art could determine whether an antibody competitively interferes with another antibody using standard methods.

By “epitope” is intended the part of an antigenic molecule to which an antibody is produced and to which the antibody will bind. Epitopes can comprise linear amino acid residues (i.e., residues within the epitope are arranged sequentially one after another in a linear fashion), nonlinear amino acid residues (referred to herein as “nonlinear epitopes”; these epitopes are not arranged sequentially), or both linear and nonlinear amino acid residues. Typically epitopes are short amino acid sequences, e.g. about five amino acids in length. Systematic techniques for identifying epitopes are known in the art and are described, for example, in U.S. Pat. No. 4,708,871. Briefly, a set of overlapping oligopeptides derived from the antigen may be synthesized and bound to a solid phase array of pins, with a unique oligopeptide on each pin. The array of pins may comprise a 96-well microtiter plate, permitting one to assay all 96 oligopeptides simultaneously, e.g., for binding to a biomarker-specific monoclonal antibody. Alternatively, phage display peptide library kits (New England BioLabs) are currently commercially available for epitope mapping. Using these methods, the binding affinity for every possible subset of consecutive amino acids may be determined in order to identify the epitope that a given antibody binds. Epitopes may also be identified by inference when epitope length peptide sequences are used to immunize animals from which antibodies are obtained.

Antigen-binding fragments and variants of the monoclonal antibodies disclosed herein are further provided. Such variants will retain the desired binding properties of the parent antibody. Methods for making antibody fragments and variants are generally available in the art. For example, amino acid sequence variants of a monoclonal antibody described herein, can be prepared by mutations in the cloned DNA sequence encoding the antibody of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (Cold Spring Harbor, N.Y.); U.S. Pat. No. 4,873,192; and the references cited therein; herein incorporated by reference. Guidance as to appropriate amino acid substitutions that do not affect biological activity of the polypeptide of interest may be found in the model of Dayhoffet al. (1978) in Atlas of Protein Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, GlyAla, ValIleLeu, AspGlu, LysArg, AsnGln, and PheTrpTyr.

In constructing variants of the antibody polypeptide of interest, modifications are made such that variants continue to possess the desired activity, i.e., similar binding affinity to the biomarker. Obviously, any mutations made in the DNA encoding the variant polypeptide must not place the sequence out of reading frame and preferably will not create complementary regions that could produce secondary mRNA structure. See EP Patent Application Publication No. 75,444.

Preferably, variants of a reference biomarker antibody have amino acid sequences that have at least 70% or 75% sequence identity, preferably at least 80% or 85% sequence identity, more preferably at least 90%, 91%, 92%, 93%, 94% or 95% sequence identity to the amino acid sequence for the reference antibody molecule, or to a shorter portion of the reference antibody molecule. More preferably, the molecules share at least 96%, 97%, 98% or 99% sequence identity. For purposes of the present invention, percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489. A variant may, for example, differ from the reference antibody by as few as 1 to 15 amino acid residues, as few as 1 to 10 amino acid residues, such as 6-10, as few as 5, as few as 4, 3, 2, or even 1 amino acid residue.

With respect to optimal alignment of two amino acid sequences, the contiguous segment of the variant amino acid sequence may have additional amino acid residues or deleted amino acid residues with respect to the reference amino acid sequence. The contiguous segment used for comparison to the reference amino acid sequence will include at least 20 contiguous amino acid residues, and may be 30, 40, 50, or more amino acid residues. Corrections for sequence identity associated with conservative residue substitutions or gaps can be made (see Smith-Waterman homology search algorithm).

The antibodies disclosed herein are selected to have specificity for MMP-26. Methods for making antibodies and for selecting appropriate antibodies are known in the art. See, for example, Celis, ed. (in press) Cell Biology & Laboratory Handbook, 3rd edition (Academic Press, New York), which is herein incorporated in its entirety by reference. In some embodiments, commercial antibodies directed to specific biomarker proteins may be used to practice the invention. The antibodies disclosed herein may be selected on the basis of desirable staining of histological samples. That is, in preferred embodiments the antibodies are selected with the end sample type (e.g., formalin-fixed, paraffin-embedded breast tumor tissue samples) in mind and for binding specificity.

In one example, antibodies directed to specific biomarkers of interest can be selected and purified via a multi-step screening process. For example, polydomas are screened to identify biomarker-specific antibodies that possess the desired traits of specificity and sensitivity. As used herein, “polydoma” refers to multiple hybridomas. The polydomas are typically provided in multi-well tissue culture plates. In the initial antibody screening step, a set of individual slides or tumor tissue microarrays comprising normal (i.e., non-cancerous) breast tissue and stage I, II, III, and IV breast tumor samples is used. Methods and equipment, such as the Chemicon™ Advanced Tissue Arrayer, for generating arrays of multiple tissues on a single slide are known in the art. See, for example, U.S. Pat. No. 4,820,504. Undiluted supernatants from each well containing a polydoma are assayed for positive staining using standard immunohistochemistry techniques. At this initial screening step, background, non-specific binding is essentially ignored. Polydomas producing positive staining are selected and used in the second phase of antibody screening.

In the second screening step, the positive polydomas are subjected to a limiting dilution process. The resulting unscreened antibodies are assayed via standard immunohistochemistry techniques for positive staining of breast tumor tissue samples with known 5-year outcomes. To do this, tissue microarrays comprising normal breast tissue, early-stage breast tumor samples with known good 5-year outcomes, early-stage breast tumor samples with known bad 5-year outcomes, normal non-breast tissue, and cancerous non-breast tissue are generated. At this stage, background staining is relevant, and the candidate polydomas that stain positive for abnormal cells (i.e., cancer cells) only are selected for further analysis to identify antibodies that differentiate good and bad outcome patient samples.

Positive-staining cultures are prepared as individual clones in order to select individual candidate monoclonal antibodies. Methods for isolating individual clones and for purifying antibodies through affinity adsorption chromatography are well known in the art. Individual clones are further analyzed to determine the optimized antigen retrieval conditions and working dilution.

One of skill in the art will recognize that optimization of staining reagents and conditions, for example, antibody titer and detection chemistry parameters, is needed to maximize the signal to noise ratio for a particular antibody. Antibody concentrations that maximize specific binding to the biomarkers of the invention and minimize non-specific binding (or “background”) can be determined. In particular embodiments, appropriate antibody titers are determined by initially testing various antibody dilutions on formalin-fixed, paraffin-embedded normal and cancerous breast tissue samples. The design of assays to optimize antibody titer and detection conditions is standard and well within the routine capabilities of those of ordinary skill in the art. Some antibodies require additional optimization to reduce background staining and/or to increase specificity and sensitivity of staining.

Furthermore, one of skill in the art will recognize that the concentration of a particular antibody used to practice the methods disclosed herein will vary depending on such factors as time for binding, level of specificity of the antibody for the biomarker protein, and method of body sample preparation. Moreover, when multiple antibodies are used in a single sample, the required concentration may be affected by the order in which the antibodies are applied to the sample, i.e., simultaneously as a cocktail or sequentially as individual antibody reagents. Furthermore, the detection chemistry used to visualize antibody binding to a biomarker of interest must also be optimized to produce the desired signal to noise ratio.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, .beta.-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material.

In regard to detection of antibody staining in the immunohistochemistry methods disclosed herein, there also exist in the art, video-microscopy and software methods for the quantitative determination of an amount of multiple molecular species (e.g., biomarker proteins) in a biological sample wherein each molecular species present is indicated by a representative dye marker having a specific color. Such methods are also known in the art as a calorimetric analysis methods. In these methods, video-microscopy is used to provide an image of the biological sample after it has been stained to visually indicate the presence of a particular biomarker of interest. Some of these methods, such as those disclosed in U.S. patent application Ser. No. 09/957,446 to Marcelpoil et al. and U.S. patent application Ser. No. 10/057,729 to Marcelpoil et al., incorporated herein by reference, disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that is “deconstructed” into its component color parts.

The methods disclosed herein can be used in conjunction with imaging systems and associated imaging software for the detection of biomarker expression.

ii. Nucleic Acid Detection

The expression of a biomarker of interest can also be detected at the nucleic acid level. Nucleic acid-based techniques for assessing expression are well known in the art and include, for example, determining the level of biomarker mRNA in a body sample. Many expression detection methods use isolated RNA. Any RNA isolation technique that does not select against the isolation of mRNA can be utilized for the purification of RNA (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The term “probe” refers to any molecule that is capable of selectively binding to a specifically intended target biomolecule, for example, a nucleotide transcript or a protein encoded by or corresponding to a biomarker, such as MMP-26. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding an MMP-26 biomarker. Hybridization of an mRNA with the probe indicates that the biomarker in question is being expressed.

In one example, the mRNA is immobilized on a solid surface and contacted with a probe, for example by running the isolated mRNA on an agarose gel and transferring the mRNA from the gel to a membrane, such as nitrocellulose. Alternatively, the probe(s) can be immobilized on a solid surface and the mRNA is contacted with the probe(s), for example, in an Affymetrix gene chip array. A skilled artisan can readily adapt known mRNA detection methods for use in detecting the level of mRNA encoded by MMP-26.

An alternative method for determining the level of biomarker mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects, biomarker expression is assessed by quantitative fluorogenic RT-PCR (i.e., the TaqMan™ System).

Biomarker expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of biomarker expression may also comprise using nucleic acid probes in solution.

In one embodiment, microarrays are used to detect biomarker expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample. Techniques for the synthesis of these arrays using mechanical synthesis methods are described in, e.g., U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. Arrays may be packaged in such a manner as to allow for diagnostics or other manipulation of an all-inclusive device. See, for example, U.S. Pat. Nos. 5,856,174 and 5,922,591 herein incorporated by reference.

In one approach, total mRNA isolated from the sample is converted to labeled cRNA and then hybridized to an oligonucleotide array. Each sample is hybridized to a separate array. Relative transcript levels may be calculated by reference to appropriate controls present on the array and in the sample.

### B. KITS

Kits for practicing the methods disclosed herein are further provided. By “kit” is intended any manufacture (e.g., a package or a container) comprising at least one reagent, e.g. an antibody, a nucleic acid probe, etc. for specifically detecting the expression of MMP-26. The kit can be promoted, distributed, or sold as a unit for performing the methods of the present invention. Additionally, the kits can contain a package insert describing the kit and methods for its use.

Kits for practicing the immunohistochemistry methods of the invention are provided. Such kits are compatible with both manual and automated immunohistochemistry techniques (e.g., cell staining) as described herein. These kits comprise at least one antibody directed to MMP-26. Chemicals for the detection of antibody binding to the biomarker, a counterstain, and a bluing agent to facilitate identification of positive staining cells are optionally provided. Alternatively, the immunochemistry kits are used in conjunction with commercial antibody binding detection systems, such as, for example the Dako Envision+ system™ and Biocare Medical's Mach 3™ system. Any chemicals that detect antigen-antibody binding can be used in the practice of the methods disclosed herein. The detection chemicals can comprise a labeled polymer conjugated to a secondary antibody. For example, a secondary antibody that is conjugated to an enzyme that catalyzes the deposition of a chromogen at the antigen-antibody binding site can be provided. Such enzymes and techniques for using them in the detection of antibody binding are well known in the art. In one embodiment, the kit comprises a secondary antibody that is conjugated to an HRP-labeled polymer. Chromogens compatible with the conjugated enzyme (e.g., DAB in the case of an HRP-labeled secondary antibody) and solutions, such as hydrogen peroxide, for blocking non-specific staining can be further provided. The kits can also comprise a counterstain, such as, for example, hematoxylin. A bluing agent (e.g., ammonium hydroxide) can be further provided in the kit to facilitate detection of positive staining cells.

Any or all of the kit reagents may be provided within containers that protect them from the external environment, such as in sealed containers. Positive and/or negative controls can be included in the kits to validate the activity and correct usage of reagents employed in accordance with the invention. Controls may include samples, such as tissue sections, cells fixed on glass slides, etc., known to be either positive or negative for the presence of the biomarker of interest. The design and use of controls is standard and well within the routine capabilities of those of ordinary skill in the art. Also disclosed are kits comprising at least one nucleic acid probe that specifically binds to a biomarker nucleic acid or fragment thereof.

### C. DEFINITIONS

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

As used herein, “subject” includes, but is not limited to, animals, plants, bacteria, viruses, parasites and any other organism or entity that has nucleic acid. The subject may be a vertebrate, more specifically a mammal (e.g., a human, horse, pig, rabbit, dog, sheep, goat, non-human primate, cow, cat, guinea pig or rodent), a fish, a bird or a reptile or an amphibian. The subject may to an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In the context of endometriosis and endometriosis cells, it is understood that a subject is a subject that has or can have endometriosis and/or endometriosis cells.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “overall survival” is defined to be survival after first treatment. For instance, long-term overall survival is for at least 5 years, more preferably for at least 8 years, most preferably for at least 10 years following surgery or other treatment.

The term “disease-free survival” as used herein is defined as a time between the first diagnosis and/or first surgery to treat a cancer patient and a first reoccurrence. For example, a disease-free survival is “low” if the cancer patient has a first reoccurrence within five years after tumor resection, and more specifically, if the cancer patient has less than about 55% disease-free survival over 5 years. For example, a high disease-free survival refers to at least about 55% disease-free survival over 5 years.

The term “endocrine therapy” as used herein is defined as a treatment of or pertaining to any of the ducts or endocrine glands characterized by secreting internally and into the bloodstream from the cells of the gland. The treatment may remove the gland, block hormone synthesis, or prevent the hormone from binding to its receptor.

The term “endocrine therapy-resistant patient” as used herein is defined as a patient receiving an endocrine therapy and lacks demonstration of a desired physiological effect, such as a therapeutic benefit, from the administration of an endocrine therapy.

The term “estrogen-receptor positive” as used herein refers to cancers that do have estrogen receptors while those breast cancers that do not possess estrogen receptors are “estrogen receptor-negative.”

The term “prognosis” is used herein to refer to the prediction of the likelihood of cancer-attributable death or progression, including recurrence, metastatic spread, and drug resistance, of a neoplastic disease, such as breast cancer. The term “prediction” is used herein to refer to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention can be used clinically to make treatment decisions by choosing the most appropriate treatment modalities for any particular patient. The predictive methods of the present invention are valuable tools in predicting if a patient is likely to respond favorably to a treatment regimen, such as surgical intervention, chemotherapy with a given drug or drug combination, and/or radiation therapy, or whether long-term survival of the patient, following surgery and/or termination of chemotherapy or other treatment modalities is likely.

The term “therapeutic benefit” as used herein refers to anything that promotes or enhances the well-being of the subject with respect to the medical treatment of his condition, which includes treatment of pre-cancer, cancer, and hyperproliferative diseases. A list of nonexhaustive examples of this includes extension of the subject's life by any period of time, decrease or delay in the neoplastic development of the disease, decrease in hyperproliferation, reduction in tumor growth, delay of metastases, reduction in cancer cell or tumor cell proliferation rate, and a decrease in pain to the subject that can be attributed to the subject's condition.

The term “therapeutically effective amount” as used herein is defined as the amount of a molecule or a compound required to improve a symptom associated with a disease. For example, in the treatment of cancer such as breast cancer, a molecule or a compound which decreases, prevents, delays or arrests any symptom of the breast cancer is therapeutically effective. A therapeutically effective amount of a molecule or a compound is not required to cure a disease but will provide a treatment for a disease. A molecule or a compound is to be administered in a therapeutically effective amount if the amount administered is physiologically significant. A molecule or a compound is physiologically significant if its presence results in technical change in the physiology of a recipient organism.

The term “treatment” as used herein is defined as the management of a patient through medical or surgical means. The treatment improves or alleviates at least one symptom of a medical condition or disease and is not required to provide a cure. The term “treatment outcome” as used herein is the physical effect upon the patient of the treatment.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### MMP-26 Proteolysis of the N-Terminal Domain of the Estrogen Receptor-β Correlates with the Survival of Breast Cancer Patients

Estrogens perform many cellular functions, including their interactions with estrogen receptors-α and -β3 (ERα and ERβ). It has been determined that the estrogen-ER complex stimulates the transcriptional activity of the MMP-26 gene promoter. It was then determined that ERβ is susceptible to MMP-26 proteolysis while ERα is resistant to the protease. MMP-26 targets the N-terminal region of ERβ coding for the divergent N-terminal A/B domain that is responsible for the ligand-independent transactivation function. As a result, MMP-26 proteolysis generates the C-terminal fragments of ERβ. Immunohistochemical analysis of tissue microarrays derived from 121 cancer patients corroborated these data and revealed an inverse correlation between the ERα-dependent expression of MMP-26 and the levels of the intact ERβ in breast carcinomas (Example 1). MMP-26 is not expressed in normal mammary epithelium. The levels of MMP-26 are strongly up-regulated in ductal carcinoma in situ (DCIS). In the course of further disease progression through stages I-III, the expression of MMP-26 decreases. In contrast to many tumor-promoting MMPs, the expression of MMP-26 in DCIS correlated with a longer patient survival. The data show the existence of an MMP-26-mediated, intracellular pathway that targets ERβ and that MMP-26, a novel and valuable cancer marker, contributes favorably to the survival of the ERα/β-positive cohort of breast cancer patients.

i. Materials and Methods

a. Chemicals and Cells.

Reagents were obtained from Sigma (St. Louis, Mo.), unless otherwise indicated. Human α1-anti-trypsin (AAT) was obtained from Calbiochem (San Diego, Calif.). A hydroxamate inhibitor GM6001 and rabbit polyclonal antibody AB1410 against the 1-12 aminoacid N-terminal sequence region of ERβ were obtained from Chemicon (Temecula, Calif.). The purified ERα and ERβ were obtained from Invitrogen (Carlsbad, Calif.). Rabbit polyclonal antibody Ab-24 against the C-terminal part of ERβ was obtained from LabVision (Fremont, Calif.). Mouse monoclonal antibody 14C8 directed against the 1-153 N-terminal sequence region of ERβ was from GeneTex (San Antonio, Tex.). The rabbit polyclonal antibody against the C-terminal part of ERβ and murine monoclonal antibody 1D5 against ERβ were purchased from Santa Cruz (Santa Cruz, Calif.) and DakoCytomation (Carpenteria, Calif.), respectively. MMP-26 and the recombinant catalytic domain of MT1-MMP were expressed in E. coli and then purified from the inclusion bodies and refolded to restore their conformation and their catalytic activity (Li et al. Cancer Res 2004 64:8657-65; Ratnikov et al. Anal Biochem 2000 286:149-55; Rozanov et al. J Biol Chem 2003 278:8257-60). Rabbit polyclonal antibody, raised against the catalytic domain of MMP-26, was prepared and affinity purified as previously described (Zhao et al. J Biol Chem 2003 278:15056-64). The total concentrations of MMP-26 and the catalytic domain of MT1-MMP were measured by absorption at 280 nm and calculated using a molar extinction coefficient of 39,000 M⁻¹ cm⁻¹ and 57,000 M⁻¹ cm⁻¹, respectively. MT1-MMP and MMP-26 were each titrated with GM6001 to determine the precise concentration of catalytically active enzymes. Breast carcinoma MCF-7 cells were obtained from ATTC. Cells were routinely maintained in DMEM medium supplemented with 10% fetal bovine serum.

b. Cleavage Assays.

AAT, ERα and ERβ (500 ng each) were co-incubated for 2 h at 37° C. with the indicated amounts of the proteases in 20 μl of 50 mM HEPES buffer, pH 6.8, buffer containing 200 mM NaCl, 10 mM CaCl₂, 20 μM ZnCl₂, and 0.01% Brij-35. The reactions were stopped by adding 2% SDS and analyzed by SDS-PAGE. The digest fragments were identified by Coomassie staining or Western blotting.

c. Lentiviral Expression of MMP-26.

The full-length MMP-26 cDNA (Marchenko et al. Biochem J 2001 356:705-18) was inserted into the SpeI-YhoI restriction sites of the pLenti6/V5-D-TOPO lentiviral vector under the control of the CMV promoter. The lentiviral vector was amplified using a complete ViraPower™ Lentiviral Expression Kit in the 293FT producer cell line according to the manufacturer's instructions (Invitrogen). The harvested viral supernatant was used to transfect MCF-7 cells. One week after transfection, the MMP-26 expression was determined by Western blotting of the total blasticidin-resistant MCF-7 cell pool. Subcloning of the cells was not used in these experiments.

d. Immunoblotting.

Cells were lysed in either 50 mM Tris-HCl buffer, pH 7.4, containing 150 mM NaCl, 1% IGEPAL, 0.25% sodium deoxycholate, 1 mM sodium vanadate, 1 mM sodium fluoride, and 1 mM EDTA, or 20 mM Tris-HCl buffer, pH 7.4, containing 150 mM NaCl, 0.1% SDS, 1% Triton X-100, 1% sodium deoxycholate, and 1% IGEPAL. The lysis buffers were supplemented with a protease inhibitor cocktail for use with mammalian cells (Sigma), and with phenylmethylsulfonyl fluoride (1 mM). Equal amounts of the total protein (approximately 40 μg of total protein per sample) were analyzed by Western blotting with the MMP-26, ERα and ERβ antibodies followed by secondary species-specific IgG conjugated with horseradish peroxidase (HRP) and a TMB/M substrate (Chemicon).

e. Immunocytochemistry.

Cells were subcultured in LabTek chamber slides. The attached cells were fixed twice for 3 min with Z-Fix (10% zinc-buffered formalin, pH 5.5) (Anatech; Battle Creek, Mich.) and then blocked for 30 min with 2% BSA and 1% normal goat serum. The slides were next incubated overnight at ambient temperature with the primary antibody diluted 1:2000-1:6000 in the DakoCytomation antibody diluent (DakoCytomation) supplemented with 1% goat normal serum. The colorimetric reaction was developed by incubating the slides with the goat, HRP-conjugated anti-rabbit antibody and a 3,3′-diaminobenzidine substrate (0.25 mg/ml in PBS supplemented with 0.05% H₂O₂). Methyl green was used for counterstaining.

For immunofluorescence staining, cells were fixed with 4% paraformaldehyde, permeabilized with 0.1% Triton X-100 and incubated for 4 h with the primary antibody diluted with PBS, supplemented with 1% fetal bovine serum and 0.1% sodium azide. The slides were then incubated for 2 h with the secondary species-specific IgG conjugated with phycoerythrin. 4′,6-Diamidino-2-phenylindole (DAPI) was used for nuclear staining. The slides were mounted in VectaShield antifading embedding medium (Vector, Burlingame, Calif.) and fluorescence-labeled cells were examined under a fluorescence microscope.

f. Patient Specimens.

Archival paraffin-embedded tissue specimens containing normal mammary epithelium (n=16), in situ breast carcinomas (n=23), and invasive breast tumors, represented by the ductal (n=103), lobular (n=15), and mucinous (n=3) histological subtypes were obtained in St. Vincent's Hospital (Dublin, Ireland). These specimens represented the residual pathological materials remaining after the diagnostic and hormone receptor determinations and were derived from women who presented in 2001 with the symptomatic stage I-III breast cancers. These samples were used for the preparation of tissue microarrays (TMAs). Human breast surgical specimens were obtained under the Institutional Review Board approval of the Department of Surgery and Pathology, University College, Dublin, Ireland. In addition, 16 normal mammary epithelium specimens, excised from surgical margins, and 4 independent normal mammary gland tissue samples were included in the TMAs. The breast cancer specimens have been fixed in 8% formalin and paraffin-embedded according to routine procedures.

g. Tissue Microarrays.

To construct high density breast cancer TMAs, each containing 140-190 specimens, two to five 1-mm (diameter) cylindrical cores were taken from the representative areas of normal tissue (one core per a patient) and of malignant tissues (two-three core per a patient) from archival paraffin blocks and arrayed into a new recipient paraffin block using a custom-built precision microarrayer (Beecher Instruments, Silver Spring, Md.). Serial sections (4 μm) of the recipient block were applied to the Superfrost-Plus glass slides (Fisher) coated with 3-aminopropyltriethoxysilane (Rentrop et al. Histochem J 1986 18:271-6).

h. Immunohistochemistry.

Following routine dewaxing, the TMA were stained with the polyclonal antibody against the recombinant catalytic domain of MMP-26 (Li et al. Cancer Res 2004 64:8657-65), the murine monoclonal antibody 1D5 to ERα (DakoCytomation) and the rabbit polyclonal antibodies AB1410 and Ab-24 against the ERβ. Staining with the primary antibody was followed by a diaminobenzidine (DAB)-based detection method employing the Envision Plus HRP system (DakoCytomation) and an automated Dako immunostainer (26). For double-labeling experiments, TMAs were stained first with the Envision Plus HRP system and a DAB substrate (brown color) and then with the second primary antibody followed by either alkaline phosphatase staining with the Vector BCIP/NBT development or the ABC-HRP system and SG chromagen (Vector, Burlingame, Calif.) (grey-black color). The slides were counterstained with Nuclear red, dehydrated and mounted with permanent mounting media. For all tissues examined, the immunostaining procedure was performed in parallel using either preimmune serum or antiserum depleted by incubation with recombinant protein immunogen to verify specificity of the results. The immunostaining results were scored according to intensity as 0, negative; 1, weak; 2, moderate; and 3, strong. The scoring of immunostaining was calculated by multiplying the percentage of immunopositive cells (0 to 100) by the staining intensity score (0/1/2/3), yielding scores ranging from 0 to 300.

i. Statistical Analysis.

Data were analyzed using the STATISTICA software package (StatSoft, Tulsa. OK). The Student's t test was applied to characterize protein distribution in normal versus malignant tissues. Differences in the distribution of variables were tested using the Pearson's χ² statistics for categorical variables and the ANOVA test for continuous variables. To perform the survival analysis, the immunostaining data were dichotomized at the median, comparing the clinical outcome for patients whose tumor immunoscores were above the median with those below the median. Breast cancer patient survival in relation to MMP-26 expression was analyzed using Kaplan-Meier curves in conjunction with the log-rank test.

ii. Results

a. MMP-26 Cleaves ERβ In Vitro.

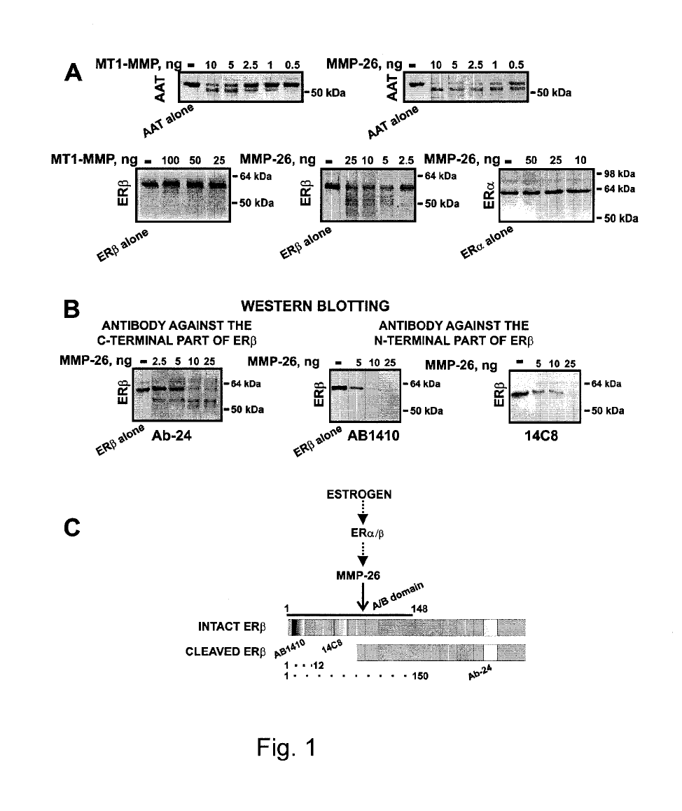

According to earlier observations, the AAT serpin is a clinically relevant protein target of proteolysis by MMP-26 (Li et al. Cancer Res 2004 64:8657-65). Consistent with these data, the catalytic amounts of MMP-26 fully proteolyzed 61 kDa AAT (the enzyme-substrate molar ratio at a range of 1:15-1:150) in 2 h in studies and generated a 55 kDa N-terminal fragment and a C-terminal fragment of approximately 6 kDa of AAT (FIG. 1A, upper right panel). The potency of MT1-MMP in cleaving AAT was lower, albeit comparable, with that of MMP-26 (FIG. 1, upper left panel). In turn, ERβ was resistant to MT1-MMP but it was sensitive to proteolysis by the catalytic amounts of MMP-26 (the enzyme-substrate molar ratio at a range of 1:30-1:60). In contrast to ERβ, ERα was not susceptible to MMP-26 (FIG. 1A, bottom panels). GM6001, and tissue inhibitors-1 and -2 of matrix metalloproteinases (TIMP-1 and TIMP-2, respectively) fully inhibited the proteolysis of ERβ by MMP-26.

The cleavage by MMP-26 transformed the 59 kDa ERβ into several digest fragments. The apparent molecular mass of the main digest fragments was in the range of 51-54 kDa, but the shorter fragments were also observed in the digest samples. To identify the relative position of the cleavage fragments within the ERβ polypeptide chain, antibodies AB1410 and 14C8 were used, and Ab-24, which recognized the N-terminal and C-terminal epitopes of ERβ, respectively. The ERβ samples were cleaved by increased amounts of MMP-26 and the digest samples were analyzed by Western blotting employing the AB1410, 14C8 and Ab-24 antibodies. As shown in FIG. 1B, the Ab-24 antibody against the C-terminal epitope recognized the intact ERβ and the digest fragments, while the Ab 1410 and 14C8 antibodies against the N-terminal epitope reacted only with the intact ERβ. These results indicate that MMP-26 proteolysis generated the stable cleavage fragments that represented the C-terminal portion of the ERβ molecule. The size difference in the apparent molecular weight between the intact ERβ and the major ERβ fragments showed that these stable, N-terminally-truncated, species are missing the first 40-60 N-terminal residues of the ERβ A/B domain and, accordingly, it appears that they are missing the functionality of the A/B domain which normally exhibits the ligand-independent AF-1 transactivation function of ERβ. FIG. 1C demonstrates, in a schematic manner, the relative positions of the antibody epitopes, the A/B domain and the MMP-26 cleavage site in the ERβ polypeptide sequence.

b. MMP-26 Cleaves Cellular ERβ

Although the available antibodies were generated to the specific sequence regions of the ERs or to the recombinant purified receptor proteins, because of the high degree of sequence homology between the ERα and ERβ, antibody specificity to the receptor subtypes was demonstrated. Using Western blotting of the purified ERα and ERβ, it was confirmed that the antibodies Ab-24, AB1410 and 14C8 to ERβ did not cross-react with ERα. It was also demonstrated that the 1D5 antibody to ERα did not recognize ERβ.

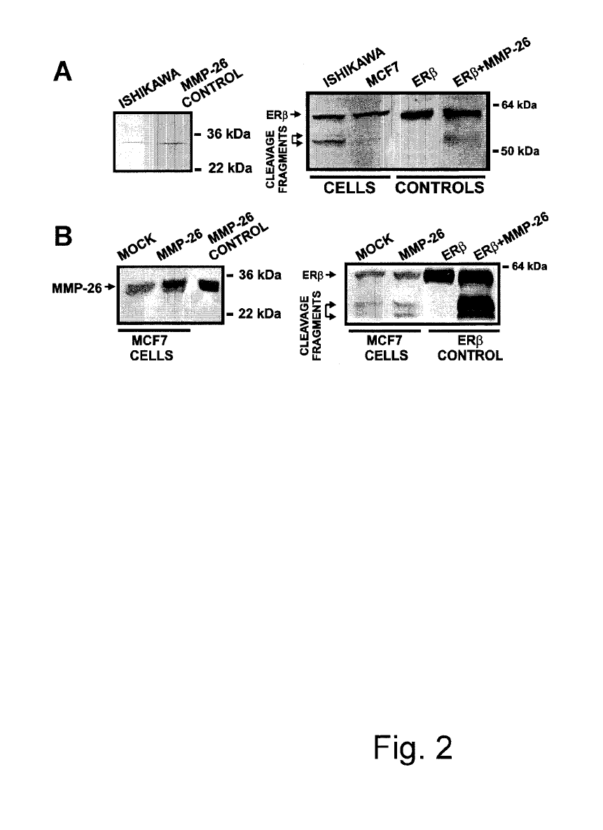

To confirm the in vitro cleavage data, MMP-26 and ERβ were evaluated by immunoblotting in endometrial carcinoma Ishikawa cells and breast carcinoma MCF-7 cells. Ishikawa and MCF7 cells were chosen because, according to earlier RT-PCR results, these cells express substantial levels of the mRNA of MMP-26 (Li et al. Cancer Res 2004 64:8657-65; 10, 21; Marchenko et al. Biochem J 2001 356:705-18; Marchenko et al. Biochem J 2002 363:253-62). A purified MMP-26 control was included along with the Ishikawa extract in Western blot analysis. In agreement with the results of RT-PCR, total cellular extracts of Ishikawa cells (FIG. 2A, left panel) and MCF-7 cells (FIG. 2B, left panel) demonstrated the presence of MMP-26. Consistent with the presence of MMP-26, the degradation products of ERβ (51-54 kDa) along with the intact 59 kDa receptor were detected by immunoblotting with the ERβ antibody Ab-24 in these cells. The molecular weight of the ERβ degradation products observed in Ishikawa and MCF-7 cells was similar to that in the control, MMP-26-cleaved, samples of the purified recombinant ERβ (FIG. 2A, middle panel, and FIG. 2B, right panel). The relative quantities of the ERβ degradation products were significantly higher in Ishikawa cells when compared to MCF-7 cells.

The cleavage of the cellular ERβ by MMP-26 was next observed. For this purpose, by using cell transfection, the expression of MMP-26 in MCF-7 cells was increased. Transfection of MCF-7 cells with a recombinant lentivirus bearing the full-length MMP-26 cDNA gene caused a noticeable increase in the MMP-26 levels (FIG. 2B, left panel). This increase correlated with an enhanced degradation of ERβ in the transfected cells when compared with mock-transfected control (FIG. 2B, right panel). The N-terminal 6 kDa cleavage fragment of ERβ was never detected in cell extracts, thus showing that this low molecular fragment was sensitive to proteolysis and that it was rapidly degraded by cellular proteinases.

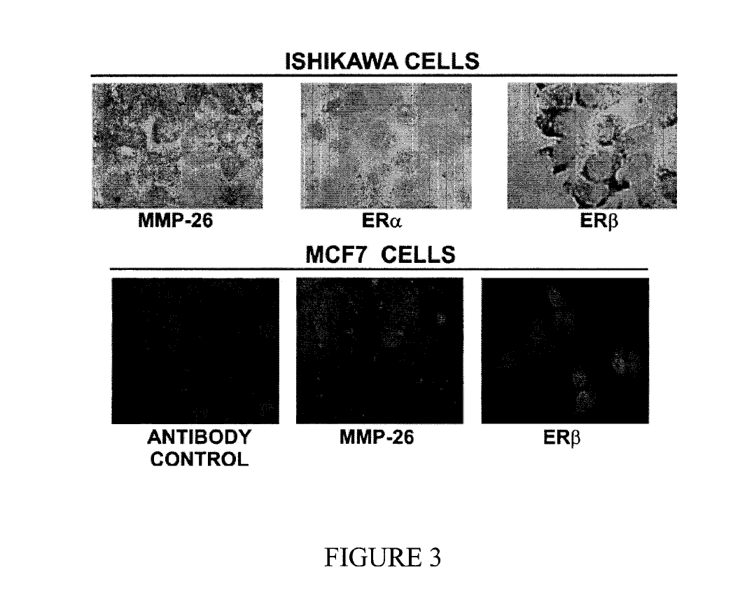

In agreement with earlier results as well as with the results of others (Mueller et al. J Biol Chem 2003 278:12255-62; Bramlett et al. J Steroid Biochem Mol Biol 2003 86:27-34; Robertson et al. J Mol Endocrinol 2002 29:125-35), immunostaining confirmed the presence of both MMP-26 and ERβ in Ishikawa cells (FIG. 3). ERα was not detected in Ishikawa cells. Consistent with the presence of the mRNA, as detected by RT-PCR and the protein as determined by Western blotting, immunofluorescence staining confirmed the expression of MMP-26 and ERβ in MCF-7 cells (FIG. 3).

c. Inverse Correlations of MMP-26 with ERβ in Breast Cancer Cells.

An immunohistochemical approach was used to analyze the expression of MMP-26, ERβ and ERα in breast tissue specimens derived from stage I-III breast cancer patients and arranged in the TMAs. The manual immunoscoring method provided highly reliable data when compared to the digital scoring systems (Cuezva et al. Cancer Res 2002 62:6674-81; Price et al. J Cell Biochem Suppl 2002 39:194-210).

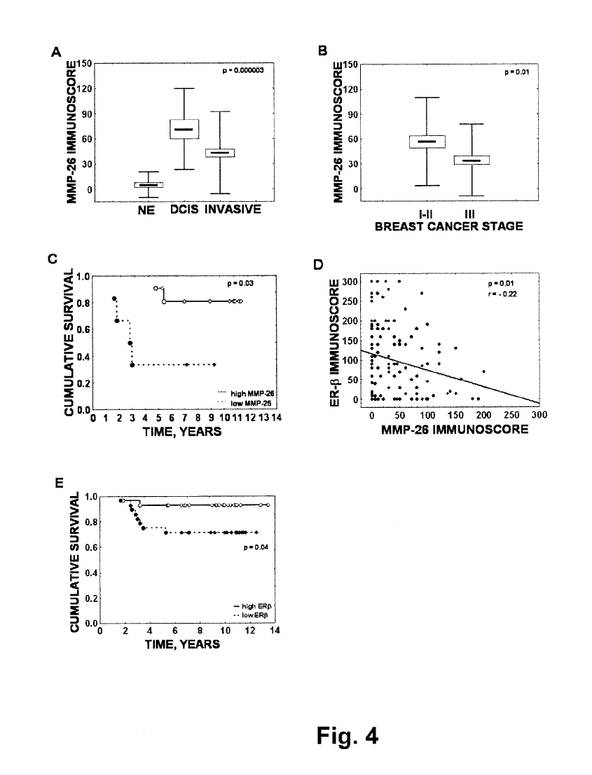

Immunostaining determined that MMP-26 immunoreactivity was high both in in situ and invasive carcinomas when compared to the normal mammary epithelium (mean immunoscores of 71±11.6, 43±11.6, and 5±2.8, respectively; p=0.000003 by ANOVA), with MMP-26 levels in in situ tumors considerably exceeding those in the other histological categories (FIG. 4A). In invasive carcinomas, high MMP-26 immunoreactivity was associated with early clinical I-II stages compared to the late stage III (p=0.01) (FIG. 4B). These unbiased observations indicated that the up-regulation of MMP-26 is an early event in the pathogenesis of breast cancer.

To correlate MMP-26 expression with the clinical outcome, the immunostaining data were dichotomized into the high versus the low protein levels, using the median immunoscore as a cut-off. In the investigated cohort, patients with the enhanced expression of MMP-26 in in situ tumors enjoyed significantly longer disease-free and overall survival when compared to patients with the low levels of MMP-26 in in situ lesions (p=0.03) (FIG. 4C).

To determine possible associations between the expression of MMP-26 and the estrogen status of the tumors, the TMAs also were stained for ERα (the antibody 1D5) and ERβ (the antibody AB1410 to the N-terminal portion of the receptor). In agreement with biochemical data which showed that ERβ is a cleavage target of MMP-26, the immunohistochemical analysis of the breast cancer TMAs revealed an inverse correlation between the MMP-26 expression and the levels of immunoreactivity of the residual intact receptor: the high immunoreactivity of MMP-26 was accompanied by a concomitant loss of ERβ in invasive adenocarcinomas (r=−0.22, p=0.01) (FIG. 4D). In agreement with other reports (Esslimani-Sahla et al. Clin Cancer Res 2004 10:5769-76; Fuqua et al. Cancer Res 1999 59:5425-8; Fuqua et al. Cancer Res 2003 63:2434-9), the results demonstrated that the presence of high levels of the intact ERβ in the ERα-positive tumors favorably correlated with a patient's survival (Kaplan-Meier analyses; FIG. 4E). Consistent with volumes of other works, high levels of the ERα immunoreactivity correlated with a longer survival of the patients in the patient cohort available to the study (p=0.01 for the overall survival and p=0.04 for the disease-free survival).

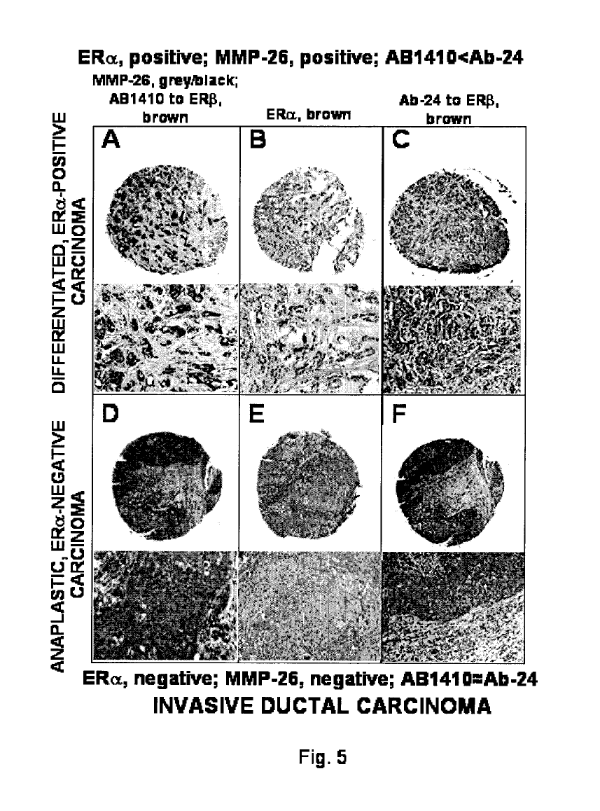

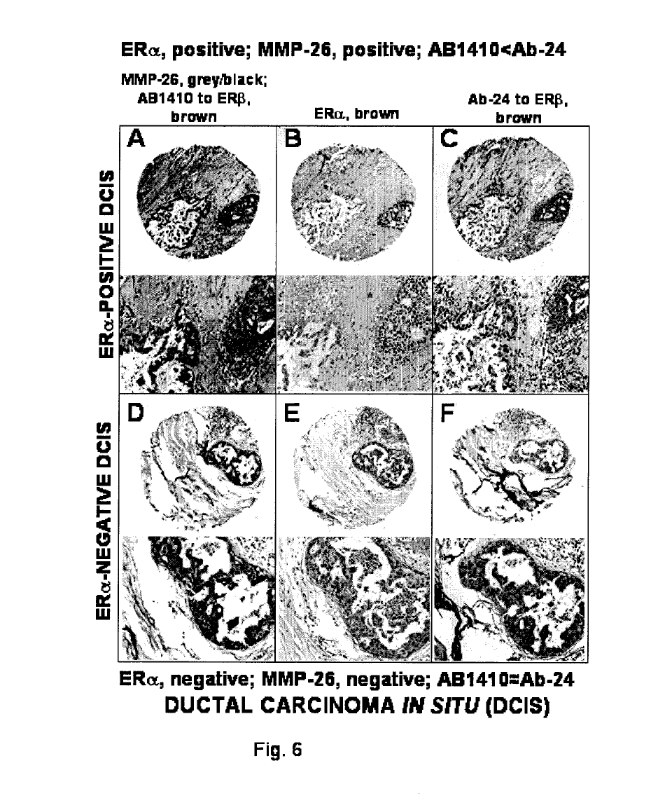

FIGS. 5 and 6 show the representative TMAs immunostained for MMP-26, ERα and ERβ in invasive ductal carcinomas and DCIS, respectively. In agreement with the regulation of MMP-26 by E2, the presence of ERα is required for the induction of the MMP-26 expression in breast carcinoma cells. In the ERα-positive/MMP-26-positive samples, the AB 1410 immunoreactivity of ERβ was low while the Ab-24 immunoreactivity of ERβ was high, thus showing the predominant presence of the proteolyzed ERβ (FIGS. 5 and 6, panels A-C). In turn, in the ERα-negative tumor specimens, the immunoreactivity of MMP-26 was minor, and the Ab-24 immunoreactivity of ERβ was similar to that of the AB1410 antibody, thus showing the predominant presence of the intact ERα (FIGS. 5 and 6, panels D-F).

iii. Discussion

E2 and its α- and β-receptors play a crucial role in the progression of hormone-dependent neoplasms, including breast cancer (Fuqua et al. Cancer Res 1999 59:5425-8; Fuqua Cancer Res 2003 63:2434-9). The ERs have been targets for breast cancer treatment for years. ERα and ERβ each play complex and distinct roles, roles which are not understood in detail, in regulating the cell response to E2. A recent comprehensive study of 305 breast cancer patients shows that low levels of ERβ predict resistance to Tamoxifen therapy in breast cancer (Hopp et al. Clin Cancer Res 2004; 10:7490-9). These data stimulated interest in the intracellular proteolytic processes, which can regulate the concentrations and the functionality of ERβ in breast carcinomas and focused attention on MMP-26, a unique matrix metalloproteinase, the expression of which is associated with carcinomas and is regulated by E2.

Consistent with the earlier structure-functional features and cellular localization of MMP-26, current results show that MMP-26, naturally expressed by the cells, was predominantly associated with the intracellular milieu (Li et al. Cancer Res 2004 64:8657-65; Marchenko et al. Int J Biochem Cell Biol 2004 36:942-56; Marchenko et al. Biochem J 2001356:705-18). According to additional results as well the observations of other authors (Park et al. J Biol Chem 2003 278:51646-53), the presence of the unorthodox PH₈₁CGVPD cysteine-switch motif in the sequence of MMP-26 stimulates the autolytic mechanism of the protease activation. The promoter of the MMP-26 gene represents the 5′-GGTCACTCTTGCCC-3′ ERE motif (nucleotides −129/−117), having a characteristic 13-bp palindromic element consisting of two 5-bp arms separated by a 3-bp spacer (Li et al. Cancer Res 2004 64:8657-65). In agreement with the presence of the ERE in the MMP-26 gene promoter, E2, via its interactions with the ERs, regulated the MMP-26 gene expression in Ishikawa cells. These results explain the association of the MMP-26 expression with hormone-regulated malignancies and MMP-26 cycling in the course of a menstrual period (Pilka et al. Ceska Gynekol 2004 69:467-71; Pilka et al. Ceska Gynekol 2004 69:262-6).

Based on these observations, it was if MMP-26 proteolysis targets the cellular ERs. In the current study, it was determined that MMP-26 proteolysis generates the N-terminally truncated receptor species of ERβ which lack the 40-60 amino acid long N-terminal fragment. In turn, ERα is resistant to MMP-26. The data indicate that MMP-26 attacks the N-terminal region of ERβ. This sequence region of ERβ represents a divergent N-terminal A/B domain that is responsible for the ligand-independent transactivation AF-1 function of the receptor (Huang et al. Mol Endocrinol 2005 19:2696-712).

Consistent with the biochemical in vitro data, endometrial carcinoma Ishikawa cells, which co-express MMP-26 with ERβ, naturally exhibit the proteolyzed form of ERβ. Following the transfection with the MMP-26 construct, the proteolyzed ERβ species was generated in breast carcinoma MCF-7 cells, which naturally express ERβ.

Having demonstrated the proteolysis of ERβ by MMP-26 in a cellular setting, an unbiased immunohistochemical analysis of the TMAs derived from 121 breast cancer patients was performed. Consistent with the estrogen-dependent induction of the MMP-26 expression, the presence of the protease was detected only in the ERα-positive specimens. The proteolytic mechanism of the ERβ regulation by MMP-26 is consistent with immunochemical data. These data indicated that the high levels of MMP-26 expression correlated with the presence of the N-terminally truncated species of ERβ, which was undetectable with the antibody to the N-end of the receptor but which were readily detectable with the antibody to intact C-end portion of the receptor. In contrast, ERα-negative and, consequently, MMP-26-negative biopsy samples exhibited the intact ERβ forms, which were identified with equal efficiency by the N-end- and the C-end targeting antibodies.

Overall, the analyses confirmed that there was an inverse correlation between the levels of MMP-26 and the levels of the intact ERβ in breast cancer biopsies. According to these observations, the expression of MMP-26 was insignificant in normal mammary epithelium. The expression of the protease was high in grade III invasive carcinomas and, especially in DCIS, while in stage III carcinomas the MMP-26 levels decreased. The data were consistent with the earlier results by Zhao et al. (Zhao et al. Cancer Res 2004 64:590-8) who demonstrated that the expression levels of both MMP-26 mRNA and protein were highest in human breast DCIS compared to other breast tissue samples. The data are also consistent with the recent report (Ahokas et al. J Invest Dermatol 2005 124:849-56) that stated that MMP-26 is expressed by laminin-5-positive keratinocytes in the migrating area during wound repair, in benign skin disorders characterized by inflammation and microdisruptions of basement membrane, and also in grades I and II squamous cell cancers. MMP-26, however, was not present in dedifferentiated grade III tumors. Based on these independent observations, it was suspected that MMP-26 is up-regulated during the early stages of cancer and then, as the cancer progresses, the levels of the enzyme decrease. It appears that MMP-26 is a part of an inflammatory response and that its presence contributes to a favorable prognosis of the disease progression. In agreement with this, an unexpected, but significant, direct correlation between the expression of MMP-26 in ductal carcinomas in situ and patients' survival was observed MMP-26, in addition to MMP-8 (Balbin et al. Nat Genet 2003 35:252-7), is the only species of MMP that demonstrated anti-tumor properties. From these perspectives, MMP-26 (matrilysin-2) is very different from MMP-7 (matrilysin-1), a structurally similar enzyme that is directly involved in tumor progression (Jiang et al. Clin Cancer Res 2005 11:6012-9; Shiomi et al. Cancer Metastasis Rev 2003; 22:145-52). It appears that the lack of MMP-26 in DCIS is an independent marker of aggressive growth of ERα/β-positive breast carcinomas.

Taken together, the data show the presence of an MMP-26-mediated, intracellular, regulatory pathway that targets ERβ in hormone-regulated malignancies. It appears that this pathway plays an important role in E2 signaling by regulating the levels and the functionality of cellular ERβ.

### E. REFERENCES

1. Egeblad M, Werb Z. New functions for the matrix metalloproteinases in cancer progression. Nat Rev Cancer 2002; 2:161-74.
2. Nagase H, Woessner J F, Jr. Matrix metalloproteinases. J Biol Chem 1999; 274:21491-4.
3. Zhao Y G, Xiao A Z, Newcomer R G, et al. Activation of pro-gelatinase B by endometase/matrilysin-2 promotes invasion of human prostate cancer cells. J Biol Chem 2003; 278:15056-64.
4. Park H I, Turk B E, Gerkema F E, Cantley L C, Sang Q X. Peptide substrate specificities and protein cleavage sites of human endometase/matrilysin-2/matrix metalloproteinase-26. J Biol Chem 2002; 277:35168-75.
5. Marchenko N D, Marchenko G N, Strongin A Y. Unconventional activation mechanisms of MMP-26, a human matrix metalloproteinase with a unique PHCGXXD cysteine-switch motif. J Biol Chem 2002; 277:18967-72.
6. Li W, Savinov A Y, Rozanov D V, et al. Matrix metalloproteinase-26 is associated with estrogen-dependent malignancies and targets alpha1-antitrypsin serpin. Cancer Res 2004; 64:8657-65.
7. Yamamoto H, Vinitketkumnuen A, Adachi Y, et al. Association of matrilysin-2 (MMP-26) expression with tumor progression and activation of MMP-9 in esophageal squamous cell carcinoma. Carcinogenesis 2004; 25:2353-60.
8. Marchenko N D, Marchenko G N, Weinreb R N, et al. Beta-catenin regulates the gene of MMP-26, a novel metalloproteinase expressed both in carcinomas and normal epithelial cells. Int J Biochem Cell Biol 2004; 36:942-56.
9. Goffin F, Munaut C, Frankenne F, et al. Expression pattern of metalloproteinases and tissue inhibitors of matrix-metalloproteinases in cycling human endometrium. Biol Reprod 2003; 69:976-84.
10. Marchenko G N, Ratnikov B I, Rozanov D V, Godzik A, Deryugina E I, Strongin A Y. Characterization of matrix metalloproteinase-26, a novel metalloproteinase widely expressed in cancer cells of epithelial origin. Biochem J 2001; 356:705-18.
11. Uria J A, Lopez-Otin C. Matrilysin-2, a new matrix metalloproteinase expressed in human tumors and showing the minimal domain organization required for secretion, latency, and activity. Cancer Res 2000; 60:4745-51.
12. de Coignac A B, Elson G, Delneste Y, et al. Cloning of MMP-26. A novel matrilysin-like proteinase. Eur J Biochem 2000267:3323-9.
13. Park H I, Ni J, Gerkema F E, Liu D, Belozerov V E, Sang Q X. Identification and characterization of human endometase (Matrix metalloproteinase-26) from endometrial tumor. J Biol Chem 2000; 275:20540-4.
14. Isaka K, Nishi H, Nakai H, et al. Matrix metalloproteinase-26 is expressed in human endometrium but not in endometrial carcinoma. Cancer 2003; 97:79-89.
15. Gustafsson J A. Estrogen receptor beta—a new dimension in estrogen mechanism of action. J Endocrinol 1999; 163:379-83.
16. Tong D, Schuster E, Seifert M, Czerwenka K, Leodolte S, Zeillinger R. Expression of estrogen receptor beta isoforms in human breast cancer tissues and cell lines. Breast Cancer Res Treat 2002; 71:249-55.
17. Pettersson K, Gustafsson J A. Role of estrogen receptor beta in estrogen action. Annu Rev Physiol 2001; 63:165-92.
18. Chegini N, Rhoton-Vlasak A, Williams R S. Expression of matrix metalloproteinase-26 and tissue inhibitor of matrix metalloproteinase-3 and -4 in endometrium throughout the normal menstrual cycle and alteration in users of levonorgestrel implants who experience irregular uterine bleeding. Fertil Steril 2003; 80:564-70.
19. Pilka R, Whatling C, Domanski H, Hansson S, Eriksson P, Casslen B. Epithelial expression of matrix metalloproteinase-26 is elevated at mid-cycle in the human endometrium. Mol Hum Reprod 2003; 9:271-7.
20. Li Q, Wang H, Zhao Y, Lin H, Sang Q A, Zhu C. Identification and specific expression of matrix metalloproteinase-26 in rhesus monkey endometrium during early pregnancy. Mol Hum Reprod 2002; 8:934-40.
21. Marchenko G N, Marchenko N D, Leng J, Strongin A Y. Promoter characterization of the novel human matrix metalloproteinase-26 gene: regulation by the T-cell factor-4 implies specific expression of the gene in cancer cells of epithelial origin. Biochem J 2002; 363:253-62.
22. Pavlaki M, Zucker S. Matrix metalloproteinase inhibitors (MMPIs): the beginning of phase I or the termination of phase III clinical trials. Cancer Metastasis Rev 2003; 22:177-203.
23. Ratnikov B, Deryugina E, Leng J, Marchenko G, Dembrow D, Strongin A. Determination of matrix metalloproteinase activity using biotinylated gelatin. Anal Biochem 2000; 286:149-55.
24. Rozanov D V, Strongin A Y. Membrane Type-1 Matrix Metalloproteinase Functions as a Proprotein Self-convertase. Expression of the latent zymogen in Pichia pastoris, autolytic activation, and the peptide sequence of the cleavage forms. J Biol Chem 2003; 278:8257-60.
25. Rentrop M, Knapp B, Winter H, Schweizer J. Aminoalkylsilane-treated glass slides as support for in situ hybridization of keratin cDNAs to frozen tissue sections under varying fixation and pretreatment conditions. Histochem J 1986; 18:271-6.
26. Krajewski S, Krajewska M, Turner B C, et al. Prognostic significance of apoptosis regulators in breast cancer. Endocr Relat Cancer 1999; 6:29-40.
27. Mueller S O, Hall J M, Swope D L, Pedersen L C, Korach K S. Molecular determinants of the stereoselectivity of agonist activity of estrogen receptors (ER) alpha and beta. J Biol Chem 2003; 278:12255-62.
28. Bramlett K S, Burris T P. Target specificity of selective estrogen receptor modulators within human endometrial cancer cells. J Steroid Biochem Mol Biol 2003; 86:27-34.
29. Robertson J A, Farnell Y, Lindahl L S, Ing N H. Estradiol up-regulates estrogen receptor messenger ribonucleic acid in endometrial carcinoma (Ishikawa) cells by stabilizing the message. J Mol Endocrinol 2002; 29:125-35.
30. Cuezva J M, Krajewska M, de Heredia M L, et al. The bioenergetic signature of cancer: a marker of tumor progression. Cancer Res 2002; 62:6674-81.
31. Price J H, Goodacre A, Hahn K, et al. Advances in molecular labeling, high throughput imaging and machine intelligence portend powerful functional cellular biochemistry tools. J Cell Biochem Suppl 2002; 39:194-210.
32. Esslimani-Sahla M, Simony-Lafontaine J, Kramar A, et al. Estrogen receptor beta (ER beta) level but not its ER beta cx variant helps to predict tamoxifen resistance in breast cancer. Clin Cancer Res 2004; 10:5769-76.
33. Fuqua S A, Schiff R, Parra I, et al. Expression of wild-type estrogen receptor beta and variant isoforms in human breast cancer. Cancer Res 1999; 59:5425-8.
34. Fuqua S A, Schiff R, Parra I, et al. Estrogen receptor beta protein in human breast cancer: correlation with clinical tumor parameters. Cancer Res 2003; 63:2434-9.
35. Hopp T A, Weiss H L, Parra I S, Cui Y, Osborne C K, Fuqua S A. Low levels of estrogen receptor beta protein predict resistance to tamoxifen therapy in breast cancer. Clin Cancer Res 2004; 10:7490-9.
36. Park H I, Jin Y, Hurst D R, et al. The intermediate S1′ pocket of the endometase/matrilysin-2 active site revealed by enzyme inhibition kinetic studies, protein sequence analyses, and homology modeling. J Biol Chem 2003; 278:51646-53.
37. Pilka R, Kudela M, Hansson S, Casslen B. [MMP-26 expression in endometrial explants treated with estradiol and progesterone]. Ceska Gynekol 2004; 69:467-71.
38. Pilka R, Kudela M, Hansson S, Casslen B. [Novel matrix metalloproteinases in cycling endometrium]. Ceska Gynekol 2004; 69:262-6.
39. Huang J, Li X, Maguire C A, Hilf R, Bambara R A, Muyan M. Binding of estrogen receptor beta to estrogen response element in situ is independent of estradiol and impaired by its amino terminus. Mol Endocrinol 2005; 19:2696-712.
40. Zhao Y G, Xiao A Z, Park H I, et al. Endometase/matrilysin-2 in human breast ductal carcinoma in situ and its inhibition by tissue inhibitors of metalloproteinases-2 and -4: a putative role in the initiation of breast cancer invasion. Cancer Res 2004; 64:590-8.
41. Ahokas K, Skoog T, Suomela S, et al. Matrilysin-2 (matrix metalloproteinase-26) is upregulated in keratinocytes during wound repair and early skin carcinogenesis. J Invest Dermatol 2005; 124:849-56.
42. Balbin M, Fueyo A, Tester A M, et al. Loss of collagenase-2 confers increased skin tumor susceptibility to male mice. Nat Genet 2003; 35:252-7.
43. Jiang W G, Davies G, Martin T A, et al. Targeting matrilysin and its impact on tumor growth in vivo: the potential implications in breast cancer therapy. Clin Cancer Res 2005; 11:6012-9.
44. Shiomi T, Okada Y. MT1-MMP and MMP-7 in invasion and metastasis of human cancers. Cancer Metastasis Rev 2003; 22:145-52.

What is claimed is:

1. A method for evaluating the prognosis of a subject with cancer, the method comprising detecting a biomarker comprising MMP-26 in the subject, wherein the presence, level, amount, or a combination, of MMP-26 is indicative of the prognosis of the subject.

2. The method of claim 1, wherein the cancer is breast cancer.

3. The method of claim 2, wherein the cancer is ductal carcinoma in situ.

4. The method of claim 2, wherein the breast cancer is ERα/β-positive.

5. The method of claim 1, wherein the level of MMP-26 is measured in the subject.

6. The method of claim 5, wherein higher levels of MMP-26 indicate a good prognosis.

7. The method of claim 1 further comprising assessing clinical information of the subject.

8. The method of claim 7, wherein the clinical information comprises tumor size, tumor grade, lymph node status, age, menopause status, chance of recurrence, disease free and overall survival rate, applied therapy strategy, status of ERα, PR and Her-2/neu status, and family history.

9. The method of claim 1, wherein the method for evaluating the prognosis of a subject with breast cancer further comprises assessing one or more additional biomarkers in the subject.

10. The method of claim 1 or 9, wherein the further biomarker is selected from the group consisting of: MYC, RB1, TP53, ATM, BAX, BRCA1, BRCA2, EGFR, ESR1, NME1, PTEN, BCL2, CCND1, CCNE1, CDK4, FGF3, FGF8, IGF2, MAPK3, PRKCA, TGFα, TGFB1, TGFB2, TGFB3, VEGF, CDK2, EGF, PCNA, BMP6, CSF1, CSF3, FGF18, TNF, IGF1, ODZ1, PLG, ESR2, IGFBP3, TSG101, AR, ERBB2, ERBB4, PRKD1, PRL, MX1, PRKCE, AKT1, BAG3, BCL2L1, PRKCZ, RAD51, XRCC3, CD34, CDH1, CTNNB1, ITGB3, PECAM1, ALB, COL4A2, INS, KLK13, MMP1, MMP9, SERPINE1, SHBG, ERBB3, PDPK1, PRKCB1, PRKCD, PRKCG, PRKCZ, PRKD2, SRC, TYK2, EGR3, FOS, JUN, NR4A1, PGR, SP1, CTSB, CTSC, CTSD, CTSE, CTSL2, PCSK6, ABCB1, ABCG2, AKAP1, CEACAM5, CYB5, CYC1, CYP19A1, GSTM1, GSTM3, KRT19, MIB1, MUC1, MUC19, VIM, CCNE2, EXT1, CCNB1, CCNB2, CDC25B, CENPF, MKI67, MYBL2, PCTK1, PSMD2, MCM6, ORC6L, RFC4, RRM2, BIRC5, CKS2, MAD2L1, SMC4L1, STK6, ESM1, FLT1, BTG2, CHPT1, IGFBP5, WISP1, BUB1, CKS2, MAPRE2, MKI67, NDRG1, BAG1, BIRC5, BNIP3, RAD21, STK3, ADM, CP, MATN3, RBP3, TFRC, CDC42BPA, CKS2, MELK, STK3, STK32B, MTMR2, EZH2, HMGB3, IVNS1ABP, KIAA1442, MCM6, MLLT10, PIR, SEC14L2, TBX3, TRIP13, BIRC5, GGH, PITRM1, UCHL5, ACADS, ALDH4A1, ALDH6A1, AP2B1, ASNS, ASPM, BBC3, BM039, C20orf103, C20orf28, C20orf46, CA9, CD68, CENPA, CIRBP, CTPS, DCK, DEGS, DEPDC1, DKFZP434B168, DKFZp762E1312, DLG7, ECT2, EGLN1, EIF2C2, ERP70, EVL, FBP1, FBXO31, FBXO5, FGD6, FLJ10134, FLJ10156, FLJ10511, FLJ10901, FLJ12150, FLJ21924, FLJ22341, FUT8, GBE1, GCN1L1, GMPS, GNAZ, GPR126, GPSM2, GRB7, HRASLS, HRB, 1HPK2, ITR, KIAA0882, KIAA1181, KIAA1217, KIAA1324, KIAA1683, KIF14, KIF21A, KIF3B, KNTC2, KRT18, LCHN, LGP2, LOC388134, LOC56901, LYRIC, M160, MCCC1, MGAT4A, MIR, MLF1IP, MRPL13, MS4A7, MYRIP, NMB, NMU, NUSAP1, ODZ3, OXCT, PALM2-AKAP2, PAQR3, PECI, PEX12, PFKP, PGK1, PIB5PA, PLEKHA1, PRAME, PRC1, PRO2000, PSMD7, PTDSS1, PTPLB, QDPR, RAB27B, RAB6B, RAI2, RAMP, RASL11B, RPS4X, RRAGD, SACS, SCUBE2, SERF1A, SLC2A3, SLC7A1, Spc25, ST7, STMN1, STX1A, SYNCRIP, TK1, TMEFF1, ER-β cleavage products, and caspase-14.

11. The method of claim 1, wherein the prognosis is used to develop a treatment strategy for the subject.

12. The method of claim 1, wherein the prognosis is used to determine disease progression in the subject.

13. A method for predicting a response of a subject with cancer to a selected treatment, the method comprising detecting a biomarker comprising MMP-26 in the subject, wherein the presence, level, amount, or a combination, of MMP-26 is indicative of a given response to the selected treatment, thereby predicting the response of the subject with cancer to the selected treatment.

14. A method of predicting the likelihood of survival of a subject with cancer comprising detecting a biomarker comprising MMP-26 in the subject, wherein the presence, level, amount, or a combination, of MMP-26 is indicative of the likelihood of survival.

15. The method of claim 14, wherein higher levels of MMP-26 predict a higher survival rate in the subject.

16. The method of claim 14, wherein the likelihood of survival is used to develop a treatment strategy for the subject.

17. The method of claim 14, wherein the survival rate is measured by the percentage of chance for five-year survival.

18. A method of treating cancer in a subject, the method comprising:
a) identifying the presence of a biomarker comprising MMP-26 in a subject;
b) determining treatment type based on the presence of MMP-26 in the subject;
c) treating the subject according to the results of step (b).

19. A method of determining the effectiveness of an anti-cancer therapy comprising:
a) obtaining a sample from a subject undergoing anti-cancer therapy, and
b) monitoring the sample for expression of MMP-26, whereby expression of MMP-26 indicates the effectiveness of the anti-cancer therapy.

20. The method of claim 19, wherein the level of expression of MMP-26 is compared with a previous sample taken from the same subject.

21. The method of claim 19, wherein the level of expression of MMP-26 is compared with a standard level.

22. The method of claim 19, wherein increasing levels of MMP-26 indicates an effective anti-cancer therapy.

23. A kit comprising an assay for measuring MMP-26 levels in a subject.
